(19) United States
(12) Patent Application Publication
(10) Pub. No.: US 2009/0176718 A1
(43) Pub. Date: Jul. 9, 2009

(54) Berry Preparations For Treatment Of Diabetes And Metabolic Syndrome

(76) Inventors: David Ribnicky, North Branch, NJ (US); Ilya Raskin, Manalapan, NJ (US)

Correspondence Address: Cozen O''Connor, 250 PARK AVENUE, NEW YORK, NY, 10177, US

(21) Appl. No.: 12/263,297

(22) Filed: Oct. 31, 2008

Related U.S. Application Data
(60) Provisional application No. 60/984,293, filed on Oct. 31, 2007.

(51) Int. Cl. A61K 31/7048; A61P 3/10

(52) U.S. Cl. 514/27

(57) ABSTRACT

The present invention provides a pharmaceutical composition for the treatment of a disordered metabolism syndrome comprising anthocyanin-rich extracts from berries. The present invention further provides a method for treating a disordered metabolism syndrome utilizing anthocyanin-rich extracts from berries.

### CROSS REFERENCES

This application claims priority to U.S. Patent Application 60/984,293, filed 31 Oct. 2007, which is incorporated in its entirety herein by reference.

### FIELD OF THE INVENTION

The present invention provides pharmaceutical compositions for the treatment of a disordered metabolism syndrome comprising anthocyanin-rich extracts prepared from berries. The present invention further provides methods for treating a disordered metabolism syndrome utilizing anthocyanin-rich extracts prepared from berries.

### BACKGROUND OF INVENTION

### 1. Disordered Metabolism

### 1.1. Diabetes

Diabetes, or diabetes mellitus, is a syndrome of disordered metabolism usually resulting from a combination of genetic and environmental factors that results in abnormally high blood sugar levels (hyperglycemia). Blood glucose levels are controlled by the hormone insulin, a product of the beta cells of the pancreas. Diabetes develops due to a diminished production of insulin (in Type 1) or resistance to the effects of insulin (in Type 2 and gestational). Both Type 1 and Type 2 diabetes lead to hyperglycemia. Hyperglycemia is a major contributor to the acute signs of diabetes, which include excessive urine production, resulting compensatory thirst and increased fluid intake, blurred vision, unexplained vision loss, lethargy, and changes in energy metabolism and the metabolism of carbohydrates, fats and proteins. Serious long-term complications of diabetes include cardiovascular disease (doubled risk), chronic renal failure, retinal damage (which can lead to blindness), nerve damage, and microvascular damage, which may cause impotence and poor wound healing. In the developed world, diabetes is the most significant cause of adult blindness in the non-elderly and the leading cause of non-traumatic amputation in adults, and diabetic nephropathy is the main illness requiring renal dialysis in the United States.

Although all forms of diabetes are treatable, there is no cure.

### 1.1.1. Types of Diabetes

There are several causes of diabetes. Some cases of diabetes are caused by the body's tissue receptors not responding to insulin, by autosomal or mitochondrial mutations leading to abnormal beta cell function, or by any disease that causes extensive damage to the pancreas.

### 1.1.2. Type 1 Diabetes

Type 1 diabetes results in a deficiency of insulin and is characterized by loss of the insulin-producing beta cells of the islets of Langerhans in the pancreas as a result of a T-cell mediated autoimmune attack. There is no known preventive measure which can be taken against Type 1 diabetes. Type 1 diabetes constitutes approximately 10% of diabetes cases in North America and Europe. Most affected people are otherwise healthy and of a healthy weight when onset occurs. Sensitivity and responsiveness to insulin are usually normal, especially in the early stages. Type 1 diabetes can affect children or adults but was traditionally termed “juvenile diabetes” because it represents a majority of the diabetes cases in children.

Type 1 diabetes primarily is treated with replacement of insulin combined with careful monitoring of blood glucose levels. Without insulin, diabetic ketoacidosis which may result in coma or death, often develops. Treatments include lifestyle adjustments (diet and exercise), subcutaneous injections of insulin, and insulin delivery via pumps or inhalation. Non-insulin treatments, including monoclonal antibodies and stem-cell based therapies, are effective in animal models. Treatment of Type 1 diabetes is life-long, but need not significantly impair normal activities, if sufficient patient training, awareness, appropriate care, discipline in testing and dosing of insulin is taken. However, treatment is burdensome for patients, insulin is replaced in a non-physiological manner, and this approach therefore is far from ideal.

### 1.1.3. Type 2 Diabetes

Type 2 diabetes mellitus is due to insulin resistance (or reduced insulin sensitivity), and reduced insulin secretion. The defective responsiveness of body tissues to insulin almost certainly involves the insulin receptor in cell membranes. In the early stage, the predominant abnormality is reduced insulin sensitivity, characterized by elevated levels of insulin in the blood. At this stage hyperglycemia can be reversed by a variety of measures and medications that improve insulin sensitivity or reduce glucose production by the liver. As the disease progresses, the impairment of insulin secretion worsens, and therapeutic replacement of insulin often becomes necessary. Several factors may have a role in the development of Type 2 diabetes including central obesity (fat concentrated around the waist in relation to abdominal organs, but not subcutaneous fat) and environmental exposures, such as bisphenol A.

Type 2 diabetes frequently remains undiagnosed as visible symptoms are typically mild, non-existent or sporadic, and usually there are no ketoacidotic episodes. Accordingly, severe long-term complications may result, including renal failure due to diabetic nephropathy, vascular disease (including coronary artery disease), vision damage due to diabetic retinopathy, loss of sensation or pain due to diabetic neuropathy, and liver damage from non-alcoholic steatohepatitis.

Type 2 diabetes usually is first treated by increasing physical activity, decreasing carbohydrate intake, and losing weight. These can restore insulin sensitivity even when the weight loss is modest (for example, around 5 kilograms), most especially when it is in abdominal fat deposits. It sometimes is possible to achieve long-term, satisfactory glucose control with these measures alone. However, the underlying tendency to insulin resistance is not lost, and so attention to diet, exercise, and weight loss must continue. The usual next step, if necessary, is treatment with oral antidiabetic drugs. Insulin production initially is only moderately impaired in type 2 diabetes. Oral medications may be used to improve insulin production, to regulate inappropriate release of glucose by the liver and attenuate insulin resistance to some extent (e.g., metformin), and to substantially attenuate insulin resistance. Oral medication eventually may fail due to further impairment of beta cell insulin secretion. At this point, insulin therapy is necessary to maintain normal or near normal glucose levels.

### 1.2. Metabolic Syndrome

The term “Metabolic Syndrome” (MeS) refers to a high-risk state for diabetes and cardiovascular disease. MeS also may be referred to as “metabolic syndrome X”, “syndrome X”, “insulin resistance syndrome”, “Reaven's syndrome”, and “CHAOS.” The term “cardiovascular disease” refers to any disorder of the heart and blood vessels that make up the cardiovascular system. Several definitions of MeS have been proposed (including those from the National Cholesterol Education Program Adult Treatment Panel III; the World Health Organization (WHO); and the American College of Endocrinology). The two major definitions for MeS are provided by the International Diabetes Federation and the revised National Cholesterol Education Program, respectively. The revised NCEP and IDF definitions of metabolic syndrome are very similar and it can be expected that they will identify many of the same individuals as having metabolic syndrome. The two differences are that IDF excludes any subject without increased waist circumference, while in the NCEP definition metabolic syndrome can be diagnosed based on other criteria and the IDF uses geography-specific cut points for waist circumference, while NCEP uses only one set of cut points for waist circumference regardless of geography. These two definitions are much closer to each other than the original NCEP and WHO definitions. Generally, MeS may be defined as having at least 3 or more of the characteristics including central obesity (waist circumference); elevated blood pressure (≧130/85 mmHg); and elevated levels of high-density lipoprotein cholesterol, triglycerides, and fasting plasma glucose. It is accepted that MeS is associated with insulin resistance and increased cardiovascular and diabetes risk.

Treatment of MeS incorporates a change of lifestyle (i.e., caloric restriction and physical activity). However, drug treatment is frequently required. Generally, the individual disorders that comprise the metabolic syndrome are treated separately. Diuretics and ACE inhibitors may be used to treat hypertension. Cholesterol drugs may be used to lower LDL cholesterol and triglyceride levels, if they are elevated, and to raise HDL levels if they are low. Drugs that decrease insulin resistance, for example, metformin and thiazolidinediones, may be utilized.

Insulin resistance is a key pathophysiologic feature of MeS and is associated strongly with co-existing cardiovascular risk factors and accelerated atherosclerosis (Haffner S. M. The insulin resistance syndrome revisited, Diabetes Care 19:275-277 (1996)). Due to the clinical consequences associated with insulin resistance (IR) in subjects with MeS and Type 2 diabetes, clinical regimens directed at increasing insulin sensitivity in vivo remain one of the most desirable goals of treatment. Although it is well established that lifestyle modification can improve IR and effectively improve many of the risk factors associated with MeS, the success of maintaining lifestyle changes in humans over a chronic period is poor. Therefore, strategies to improve IR by pharmacological means have represented the traditional approach for clinical medicine (Davidson, M. B., Diabetes Mellitus: diagnosis and treatment 4th edition, W.B. Saunders Company, Philadelphia (1998)).

### 2. Phytomedical Treatment

### 2.1. Blueberries

Blueberries are flowering plants in the genus Vaccinium, sect. Cyanococcus. The species are native only to North America. They are shrubs varying in size from 10 cm tall to 4 m tall; the smaller species are known as “lowbush blueberries” (synonymous with “wild”), and the larger species as “highbush blueberries”. The leaves may be either deciduous or evergreen, ovate to lanceolate, and from 1-8 cm long and 0.5-3.5 cm broad. The flowers are bell-shaped, white, pale pink or red, sometimes tinged greenish.

The fruit is a false berry of 5 mm to 16 mm in diameter with a flared “crown” at the end; it is pale-greenish at first, then reddish-purple, and finally indigo on ripening. It has have a sweet taste when mature, with variable acidity.

Blueberries and related fruits are nutritious and have been shown to provide benefit for a wide variety of conditions.

### 2.2. Nutrients and Phytochemicals

Blueberries have a diverse range of micronutrients, with high levels of the essential dietary mineral manganese, vitamin B6, vitamin C, vitamin K and dietary fiber. One serving provides a relatively low glycemic load score of 4 out of 100 per day.

Blueberries, especially in wild species, contain anthocyanins, other antioxidant pigments and various phytochemicals that may have therapeutic properties. Studies have shown that blueberry anthocyanins, proanthocyanidins, resveratrol, flavonols, and tannins inhibit mechanisms of cancer cell development and inflammation in vitro. Similar to red grape, some blueberry species contain in their skins significant levels of resveratrol, a phytochemical that may have anti-cancer properties. Although studies have focused on utilizing the highbush cultivar of blueberries (V. corymbosum), the content of polyphenol antioxidants and anthocyanins in lowbush (wild) blueberries (V. angustifolium) exceeds values found in highbush species.

Blueberries contain powerful antioxidants that may neutralize free radicals that cause neurodegenerative disease, cardiovascular disease, and cancer (Kraft et al., 2005, Chemopreventive potential of wild lowbush blueberry fruits in multiple stages of carcinogenesis, Journal of Food Science, 70(3) 159-166). European blueberries and their relatives are thought to improve vision and prevent macular degeneration (Murray, 1997, Bilberry (Vaccinium myrtillus), American Journal of Natural Medicine, 4(1) 17-21;). Additionally, blueberries may improve digestion, reduce colon inflammation and promote urinary tract health (PDR for Herbal Medicine, Third Edition, Thomson PDR publisher, Montvale N.J. 2004).

Some studies indicate consumption of blueberries (and similar berry fruits including cranberries) may alleviate the cognitive decline occurring in Alzheimer's disease and other conditions of aging. Further, feeding blueberries to animals may lower brain damage in experimental stroke. Some studies also may indicate that consumption of blueberries may help prevent urinary tract infections. Other animal studies indicate that blueberry consumption lowers cholesterol and total blood lipid levels, possibly affecting symptoms of heart disease. Additional research has shown that blueberry consumption may alter glycosaminoglycans, which are vascular cell components affecting control of blood pressure.

### 2.3. Leaves

Studies have indicated that the leaves of blueberries (Vaccinium angustifolium) or bilberries (Vaccinium mytillus) are useful for lowering blood glucose in certain animal models. These studies have shown limited hypoglycemic activity in humans (Watson, 1928, Some observations on the effect of blueberry leaf extract in diabetes mellitus, The Canadian Medical Association Journal, 19(2):166-171). Studies suggest the active compound responsible for the hypoglycemic activity in humans is a flavonoid, specifically myrtillin. Other compounds in the extract of the leaf include compounds closely related to myrtillin as well as other flavanoids, such as quercetin, and a caffeic acid derivative, such as chlorogenic acid. The constituents of the leaf are very different from the constituents of the fruit, which are comprised of different flavanoids and anthocyanins.

### 2.4. Flavonoids

The term “flavonoid” refers to a class of plant secondary metabolites. According to the IUPAC nomenclature, flavonoids may be classified into: flavonoids, derived from 2-phenylchromen-4-one (2-phenyl-1,4-benzopyrone) structure; isoflavonoids, derived from 3-phenylchromen-4-one (3-phenyl-1,4-benzopyrone) structure; and neoflavonoids, derived from 4-phenylcoumarine (4-phenyl-1,2-benzopyrone) structure. Most studies of flavonoids have focused on their antioxidant activity.

Flavonoids are synthesized (in situ) by the phenylpropanoid metabolic pathway in which the amino acid phenylalanine is used to produce 4-coumaroyl-CoA. This may be combined with malonyl-CoA to yield the true backbone of flavonoids, a group of compounds called “chalcones,” which contain two phenyl rings. Conjugate ring-closure of chalcones results in the familiar form of flavonoids, the three-ringed structure of a flavone. The metabolic pathway continues through a series of enzymatic modifications to yield flavanones, to yield dihydroflavonols, to yield anthocyanins. Many products are formed along this pathway including the flavonols, flavan-3-ols, proanthocyanidins (tannins) and a host of other polyphenolics.

Flavonoids are distributed widely in plants and fulfill many functions, including producing yellow or red/blue pigmentation in flowers and protecting the plant from attack by microbes and insects. The widespread distribution of flavonoids, their variety and their relatively low toxicity compared to other active plant compounds (for instance alkaloids) mean that many animals, including humans, ingest significant quantities in their diet. Experimental data suggests that flavonoids have an inherent ability to modify the body's reaction to allergens, viruses, and carcinogens, and that have provided evidence of anti-allergic, anti-inflammatory, anti-microbial and anti-cancer activity. Over 5000 naturally occurring flavonoids have been characterized from various plants. They have been classified according to their chemical structure, and are usually subdivided into the following subgroups: flavones (flavone; flavonol or 3-hydroxyflavone; flavanone; flavanonol or 3 hydroxyflavanone or 2,3-dihydroflavonol); isoflavones (for example, genistein, daidzein, glycitein); flavan-3-ols (flavanols; such as: catechins and epicatechins); and anthocyanidins. Anthocyandins are the aglycones of anthocyanins, and include cyanidin, delphinidin, malvidin, pelaronidin, peonidin, and petunidin.

### 2.4.1. Anthocyanins

Anthocyanins are water-soluble vaucolar flavonoid pigments that may appear red, purple or blue according to pH. Anthocyanins occur in tissues of higher plants and provide color in leaves, stems, roots, flowers and fruits. In photosynthetic tissues (such as leaves and some stems), anthocyanins protect cells from high-light damage by absorbing blue-green and UV light, thereby protecting the tissues from photoinhibition. Without being limited by theory, anthocyanins also may signal unpalatability, since anthocyanin synthesis often coincides with synthesis of unpalatable phenolic compounds. Anthocyanins also act as powerful antioxidants. However, it is not clear whether anthocyanins can significantly contribute to scavenging of free-radicals produced through metabolic processes in leaves, since they are located in the vacuole, and thus, are separated spatially from metabolic reactive oxygen species.

Plants rich in anthocyanins are Vaccinium species, such as blueberry, cranberry and bilberry, Rubus berries including black raspberry, red raspberry and blackberry, blackcurrant, cherry, eggplant peel, black rice, Concord grape and muscadine grape, red wine, red cabbage and violet petals. Anthocyanins are less abundant in banana, asparagus, pea, fennel, pear and potato, and may be totally absent in certain cultivars of green gooseberries. High amounts of anthocyanins are found in the seed coat of black soybean (Glycine max L. Merr.) (2000 mg/100 g) and in skins and pulp of black chokeberry (Aronia melanocarpa L.) (1480 mg/100 g).

The anthocyanins (anthocyanidins with sugar group) are mostly 3-glucosides of the anthocyanidins. The anthocyanins are subdivided into the sugar-free anthocyanidin aglycones and the anthocyanin glycosides. Over 550 anthocyanins have been reported. Anthocyanins often are used as a pH indicator, as they change from red in acids to blue in bases due to differences in chemical structure that occur in response to changes in pH.

FIG. 1 shows the basic structure of anthocyanidins:

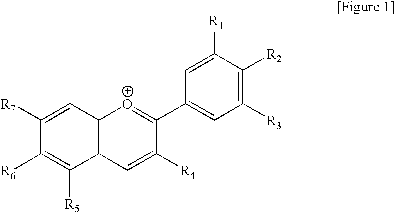

Anthocyanidins include: aurantinidin wherein R1 and R3 are —H, and R2, R4, R5, R6 and R7 are —OH; cyanidin wherein R3 and R6 are —H and R1, R2, R4, R5 and R7 are —OH; delphinidin wherein R6 is —H, and R1, R2, R3, R4, R5 and R7 are —OH; europinidin wherein R1 and R5 are —OCH3, and R2, R3, R4 and R7 are —OH, and R6 is —H; luteolinidin wherein R1, R2, R5 and R7 are —OH, and R3, R4 and R6 are —H; pelargonidin wherein R1, R3 and R6 are —H, and R2, R4, R5 and R7 are —OH; malvidin wherein R1 and R3 are —OCH3, and R2, R4, R5 and R7 are —OH, and R6 is —H; peonidin wherein R1 is —OCH3, R2, R4, R5 and R7 are —OH, R3 and R6 are —H; petunidin wherein R1, R2, R4, R5 and R7 are —OH; R3 is —OCH3, and R6 is —H; and rosinidin wherein R1 and R7 are —OCH3, R2, R4 and R5 are —OH, and R3 and R6 are —H.

Anthocyanins are powerful antioxidants in vitro. This antioxidant property may be conserved even after the plant, which produced the anthocyanin, is consumed by another organism, possibly explaining why fruits and vegetables with colorful skins and pulp are considered nutritious.

However, it has not yet been demonstrated scientifically that anthocyanins are beneficial to human health. Ongoing research purports to show that anthocyanins may have an effect against cancer, inflammation, aging and bacterial infections. The preparations of blueberry fruit have not been evaluated as potential hypoglycemic agents.

Disordered metabolism syndromes, such as diabetes and metabolic syndrome, cause significant morbidity and mortality and have high economic costs. The present invention addresses treatment of these syndromes.

### SUMMARY OF THE INVENTION

According to one aspect, the present invention provides a method for treating at least one symptom of a disordered metabolism syndrome in a subject, the method comprising the steps: (a) isolating a disordered metabolism syndrome modifying agent derived from a berry comprising an anthocyanin-rich extract; (b) administering a therapeutically effective amount of a composition comprising (i) the isolated hyperglycemia-modifying agent to a subject in need thereof and (ii) a pharmaceutically acceptable carrier; thereby treating the symptoms of the disordered metabolism syndrome. According to one embodiment, the disordered metabolism syndrome is diabetes. According to another embodiment, the metabolic syndrome. According to another embodiment, the berry is a blueberry. According to another embodiment, the blueberry is a wild lowbush blueberry. According to another embodiment, the blueberry is a fruit of the plant genus Vaccinium. According to another embodiment, the blueberry is a fruit of the plant Vaccinium angustfolium. According to another embodiment, the berry is a maqui berry. According to another embodiment, the maqui berry is a fruit of the plant genus Aristotelia. According to another embodiment, the maqui berry is a fruit of the plant Aristotelia chilensis. According to another embodiment, the berry is an elderberry from the plant genus Sambucus. According to another embodiment, the berry is a chokeberry from the plant genus Aronia. According to another embodiment, the berry is a blackcurrant from the plant genus Ribes. According to another embodiment, the berry is a blackcurrant from the plant Ribes nigrum. According to another embodiment, the therapeutically effective amount is from about 0.001 mg/kg body weight to about 10 g/kg body weight. According to another embodiment, the anthocyanini-rich extract comprises at least one of delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; and malvidin-6-acetyl-3-glucoside. According to another embodiment, the anthocyanini-rich extract comprises at least one of delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; and cyanidin 3-glucoside. According to another embodiment, the anthocyanin-rich extract is an aqueous extract. According to another embodiment, the anthocyanin-rich extract comprises at least one compound of the structure:

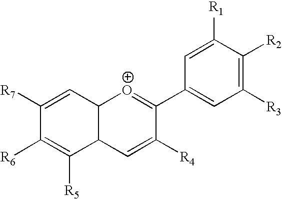

wherein R₁, R₂, R₃, R₄, R₅, R₆ and R₇ each independently is —H, —OH, —OCH₃, a monosaccharide, a disaccharide or a polysaccharide. According to another embodiment, R₁ and R₃ are —H; and R₂, R₄, R₅, R₆ and R₇ are —OH. According to another embodiment, R₃ and R₆ are —H and R₁, R₂, R₄, R₅ and R₇ are —OH. According to another embodiment, R₆ is —H, and R₁, R₂, R₃, R₄, R₅ and R₇ are —OH. According to another embodiment, R₁ and R₅ are —OCH₃, and R₂, R₃, R₄ and R₇ are —OH, and R₆ is —H. According to another embodiment, R₁, R₂, R₅ and R₇ are —OH, and R₃, R₄ and R₆ are —H. According to another embodiment, R₁, R₃ and R₆ are —H, and R₂, R₄, R₅ and R₇ are —OH. According to another embodiment, R₁ and R₃ are —OCH₃, and R₂, R₄, R₅ and R₇ are —OH, and R₆ is —H. According to another embodiment, R₁ is —OCH₃, R₂, R₄, R₅ and R₇ are —OH, R₃ and R₆ are —H. According to another embodiment, R₁, R₂, R₄, R₅ and R₇ are —OH; R₃ is —OCH₃, and R₆ is —H. According to another embodiment, R₁ and R₇ are —OCH₃, R₂, R₄ and R₅ are —OH, and R₃ and R₆ are —H. According to another embodiment, the pharmaceutically acceptable carrier is an esterified glyceride. According to another embodiment, the esterified glyceride comprises at least one of polyethylene glycol or fatty acids. According to another embodiment, the method decreases hyperglycemia by from about 5% percent to about 75% relative to a healthy subject. According to another embodiment, the composition comprises a nutritional supplement. According to another embodiment, the composition comprises a comestible. According to another embodiment, the comestible is a beverage. According to another embodiment, the hyperglycemia-modifying agent is isolated from the berry by extracting a berry homogenate with a polar solvent. According to another embodiment, the hyperglycemia-modifying agent is soluble in a polar solvent. According to another embodiment, the composition is administered orally, buccally, parenterally, topically, by inhalation or insufflation, or rectally. According to another embodiment, the composition is a pharmaceutical composition.

According to another aspect, the present invention provides a composition for the treatment of a disordered metabolism syndrome comprising (a) a therapeutically effective amount of a disordered metabolism syndrome modifying agent derived from a berry comprising an anthocyanin-rich extract; and (b) a pharmaceutically acceptable carrier. According to one embodiment, the berry is a blueberry. According to another embodiment, the blueberry is a wild lowbush blueberry. According to another embodiment, the blueberry is a fruit of the plant genus Vaccinium. According to another embodiment, the blueberry is a fruit of the plant Vaccinium angustfolium. According to another embodiment, the berry is a maqui berry. According to another embodiment, the maqui berry is a fruit of the plant genus Aristotelia. According to another embodiment, the maqui berry is a fruit of the plant Aristotelia chilensis. According to another embodiment, the berry is an elderberry from the plant genus Sambucus. According to another embodiment, the berry is a chokeberry from the plant genus Aronia. According to another embodiment, the berry is a blackcurrant from the plant genus Ribes. According to another embodiment, the berry is a blackcurrant from the plant Ribes nigrum. According to another embodiment, the therapeutically effective amount is from about 0.001 mg/kg body weight to about 10 g/kg body weight. According to another embodiment, the anthocyanin-rich extract comprises at least one of delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; and malvidin-6-acetyl-3-glucoside. According to another embodiment, the anthocyanin-rich extract comprises at least one of delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; and cyanidin 3-glucoside. According to another embodiment, the anthocyanin-rich extract is an aqueous extract. According to another embodiment, the anthocynanin-rich extract comprises at least one compound of the structure:

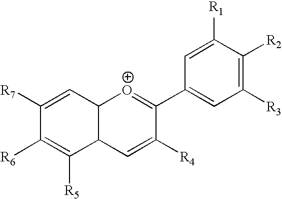

wherein R₁, R₂, R₃, R₄, R₅, R₆ and R₇ each independently is —H, —OH, —OCH₃, a monosaccharide, a disaccharide or a polysaccharide. According to another embodiment, R₁ and R₃ are —H; and R₂, R₄, R₅, R₆ and R₇ are —OH. According to another embodiment, R₃ and R₆ are —H and R₁, R₂, R₄, R₅ and R₇ are —OH. According to another embodiment, R₆ is —H, and R₁, R₂, R₃, R₄, R₅ and R₇ are —OH. According to another embodiment, R₁ and R₅ are —OCH₃, and R₂, R₃, R₄ and R₇ are —OH, and R₆ is —H. According to another embodiment, R₁, R₂, R₅ and R₇ are —OH, and R₃, R₄ and R₆ are —H. According to another embodiment, R₁, R₃ and R₆ are —H, and R₂, R₄, R₅ and R₇ are —OH. According to another embodiment, R₁ and R₃ are —OCH₃, and R₂, R₄, R₅ and R₇ are —OH, and R₆ is —H. According to another embodiment, R₁ is —OCH₃, R₂, R₄, R₅ and R₇ are —OH, R₃ and R₆ are —H. According to another embodiment, R₁, R₂, R₄, R₅ and R₇ are —OH; R₃ is —OCH₃, and R₆ is —H. According to another embodiment, R₁ and R₇ are —OCH₃, R₂, R₄ and R₅ are —OH, and R₃ and R₆ are —H. According to another embodiment, the pharmaceutically acceptable carrier is an esterified glyceride. According to another embodiment, the esterified glyceride comprises at least one of polyethylene glycol or fatty acids. According to another embodiment, the composition is a pharmaceutical composition. According to another embodiment, the method decreases hyperglycemia from about 5% to about 75% relative to a healthy subject. According to another embodiment, the composition comprises a nutritional supplement. According to another embodiment, the composition comprises a comestible. According to another embodiment, the comestible is a beverage. According to another embodiment, the disordered metabolism syndrome modifying agent is isolated from the berry by extracting a berry homogenate with a polar solvent. According to another embodiment, the disordered metabolism syndrome modifying agent is soluble in a polar solvent. According to another embodiment, the composition is administered orally, buccally, parenterally, topically, by inhalation or insufflation, or rectally.

### BRIEF DESCRIPTION OF THE DRAWINGS

FIG. 1A shows the results of an HPLC-photodiode array detection (PDA) of blueberry crude extract measured at 520 nm.

FIG. 1B shows the results of an HPLC-PDA of blueberry crude extract measured at 280 nm.

FIG. 2A shows the results of an HPLC-PDA of anthocyanin rich fraction 1 measured at 520 nm.

FIG. 2B shows the results of an HPLC-PDA of anthocyanin rich fraction 1 measured at 280 nm.

### DETAILED DESCRIPTION

The present invention provides pharmaceutical compositions for the treatment of disordered metabolism syndromes comprising anthocyanin-rich extracts from berries. The present invention further provides methods for treating a disordered metabolism syndrome utilizing anthocyanin-rich extracts from berries.

The term “disordered metabolism syndrome” as used herein refers to a disturbed, irregular, abnormal, defective, changed, mutated, nonfunctional or afflicted biological or physical process or processes in an organism by which its material substance is produced, maintained, and destroyed, and by which energy is made available; or any process of organic functioning or operating that is dysfunctional, abnormal, or defective. Disordered metabolism syndromes include, for example, but are not limited to, diabetes, diabetes mellitus, Type 1 diabetes mellitus, Type 2 diabetes mellitus, juvenile diabetes, gestational diabetes, metabolic syndrome, metabolic syndrome X, syndrome X, insulin resistance syndrome, Reaven's syndrome, CHAOS, cardiovascular disease, and hypertension.

The term “symptom” as used herein refers to a phenomenon that arises from and accompanies a particular disease or disorder and serves as an indication of it.

The term “therapeutic agent” as used herein refers to a drug, molecule, nucleic acid, protein, metabolite, composition or other substance that provides a therapeutic effect. The term “active” as used herein refers to the ingredient, component or constituent of the compositions of the present invention responsible for the intended therapeutic effect. The terms “therapeutic agent” and “active agent” are used interchangeably herein. The active agent may be, for example, but not limited to, at least one of a flavonoid or an anthocyanin, or a derivative or variant thereof, an anthocyanin of blueberry such as, for example, delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; malvidin-6-acetyl-3-glucoside (Prior, R, et al. 2001), or an anthocyanin of maqui berries such as, for example, delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; cyanidin 3-glucoside, and combinations thereof.

The term “therapeutically effective amount” or “amount effective” of one or more of the active agents is an amount that is sufficient to provide the intended benefit of treatment. An effective amount of the active agent that can be employed ranges from generally about 0.001 mg/kg body weight to about 10 g/kg body weight. However, dosage levels are based on a variety of factors, including the type of disorder, the age, weight, sex, medical condition of the subject, the severity of the condition, the route of administration, and the particular active agent employed. Thus the dosage regimen may vary widely, but can be determined routinely by a physician using standard methods.

The term “modify” as used herein means to change, vary, adjust, temper, alter, affect or regulate to a certain measure or proportion in one or more particulars.

The term “modifying agent” as used herein refers to a substance, composition, extract, botanical ingredient, botanical extract, botanical constituent, therapeutic component, active constituent, therapeutic agent, drug, metabolite, active agent, protein, non-therapeutic component, non-active constituent, non-therapeutic agent, or non-active agent that reduces, lessens in degree or extent, or moderates the form, symptoms, signs, qualities, character or properties of a condition, state, disorder, disease, symptom or syndrome.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “drug” as used herein refers to a therapeutic agent or any substance used in the prevention, diagnosis, alleviation, treatment, or cure of disease.

The term “treat” or “treating” as used herein refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in subjects that have previously had the disorder(s); and (e) limiting recurrence of symptoms in subjects that were previously symptomatic for the disorder(s).

The term “reduce” or “reducing” as used herein refers to limit occurrence of the disorder in individuals at risk of developing the disorder.

The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally.

The term “parenteral” as used herein refers to introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord or under the arachnoid membrane of the brain), intrasternal injection, or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

The term “topical” refers to administration of a composition at, or immediately beneath, the point of application. The phrase “topically applying” describes application onto one or more surfaces(s) including epithelial surfaces. Although topical administration, in contrast to transdermal administration, generally provides a local rather than a systemic effect, the terms “topical administration” and “transdermal administration” as used herein, unless otherwise stated or implied, are used interchangeably.

The term “disease” or “disorder”, as used herein, refers to an impairment of health or a condition of abnormal functioning. The term “syndrome,” as used herein, refers to a pattern of symptoms indicative of some disease or condition. The term “injury,” as used herein, refers to damage or harm to a structure or function of the body caused by an outside agent or force, which may be physical or chemical. The term “condition”, as used herein, refers to a variety of health states and is meant to include disorders or diseases caused by any underlying mechanism or disorder, injury, and the promotion of healthy tissues and organs.

The term “modulate” as used herein means to regulate, alter, adapt, or adjust to a certain measure or proportion.

The terms “subject” or “individual” or “patient” are used interchangeably to refer to a member of an animal species of mammalian origin, including humans.

The term “gavage” as used herein refers to introduction of nutritive material into the stomach by means of a tube, or forced feeding, as by a flexible tube and a force pump.

The term “alkyl” as used herein refers to a straight or branched chain optionally substituted hydrocarbon having from one to 10 carbon atoms. Examples of “alkyl” as used herein include, but are not limited to, methyl, ethyl, isopropyl, n-butyl, t-butyl and the like.

The term “chalcone” refers to an aromatic ketone that forms the central core for a variety of important biological compounds. Chalcones are intermediates in the biosynthesis of flavonoids. Chalcone preparation may be by methods known to those of skill in the art. For example, chalcones may be prepared by an aldol condensation between a benzaldehyde and an acetophenone in the presence of a sodium hydrooxide as a catalyst. The reaction between substitued benzaldehydes and acetophones also has been demonstrated.

The term “polar solvent” as used herein refers to a compound, such as, but not limited to, water, alcohol or liquid ammonia, that is composed of polar molecules. Polar solvents can dissolve ionic compounds or covalent compounds that ionize.

The term “extracting” as used herein refers to the process of drawing out, withdrawing, distilling or otherwise separating one substance from another by a chemical or physical process. The extract can be a solid, viscid, or liquid substance extracted from a plant, drug, or the like, containing its essence in concentrated form. An “anthocyanin-rich extract” refers to a preparation from any plant or part thereof, such as, for example, but not limited to, berries, blueberries or maqui berries, containing concentrations of anthocyanins, anthocyanidins and/or other related compounds greater than the naturally occurring concentrations of the compounds within the plant or part thereof, from which the extract is produced. As a non-limiting example, an extract containing concentrations of anthocyanins, anthocyanidins and/or other related compounds at least about 1% greater, about 2% greater, about 3% greater, about 4% greater, about 5% greater, about 10% greater, about 15% greater, about 20% greater, about 25% greater, about 30% greater, about 35% greater, about 40% greater, about 45% greater, about 50% greater, about 55% greater, about 60% greater, about 65% greater, about 70% greater, about 75% greater, about 80% greater, about 85% greater, about 90% greater, about 95% greater, than that of the naturally occurring concentrations of the compounds within the plant or part thereof, from which the extract is produced may be considered an anthocyanin-rich extract. As a non-limiting example, an extract containing concentrations of anthocyanins, anthocyanidins and/or other related compounds at least about 1-fold greater, about 2-fold greater, about 3-fold greater, about 4-fold greater, about 5-fold greater, about 6-fold greater, about 7-fold greater, about 8-fold greater, about 9-fold greater, about 10-fold greater, about 20-fold greater, about 30-fold greater, about 40-fold greater, about 50-fold greater, about 100-fold greater, about 500-fold greater, about 103-fold greater, about 106-fold greater, or about 109-fold greater than that of the naturally occurring concentrations of the compounds within the plant or part thereof, from which the extract is produced may be considered an anthocyanin-rich extract.

According to one aspect, the present invention provides a pharmaceutical composition for the treatment of a disordered metabolism syndrome comprising a therapeutically effective amount of an anthocyanin-rich extract from berries and a pharmaceutically acceptable carrier.

According to one embodiment, the disordered metabolism syndrome is diabetes mellitus. According to another embodiment, the disordered metabolism syndrome is diabetes. According to another embodiment, the disordered metabolism syndrome is Type 1 diabetes mellitus. According to another embodiment, the disordered metabolism syndrome is Type 2 diabetes mellitus. According to another embodiment, the disordered metabolism syndrome is juvenile diabetes. According to another embodiment, the disordered metabolism syndrome is gestational diabetes. According to another embodiment, the disordered metabolism syndrome is metabolic syndrome. According to another embodiment, the disordered metabolism syndrome is metabolic syndrome X. According to another embodiment, the disordered metabolism syndrome is syndrome X. According to another embodiment, the disordered metabolism syndrome is insulin resistance syndrome. According to another embodiment, the disordered metabolism syndrome is Reaven's syndrome. According to another embodiment, the disordered metabolism syndrome is CHAOS.

According to another embodiment, the berries are blueberries. In some such embodiments, the blueberries are wild lowbush blueberries. In some such embodiments, the blueberries are of Vaccinium angustifolium A. In another embodiment, the berries are maqui berries. In some such embodiments, the berries are of Aristotelia chilensis. According to another embodiment, the blueberry is a fruit of the plant genus Vaccinium. According to another embodiment, the maqui berry is a fruit of the plant genus Aristotelia. According to another embodiment, the berry is an elderberry from the plant genus Sambucus. According to another embodiment, the berry is a chokeberry from the plant genus Aronia. According to another embodiment, the berry is a blackcurrant from the plant genus Ribes. According to another embodiment, the berry is a blackcurrant from the plant Ribes nigrum.

According to another embodiment, the anthocyanin-rich extract comprises anthocyanins from blueberries. According to some such embodiments, the anthocyanin compounds are isolated from blueberries. According to another embodiment, the compounds are isolated from dried leaves of the blueberry. According to some such embodiments, the blueberries are wild lowbush blueberries. According to some such embodiments, the blueberries are of Vaccinium angustifolium A. Such compounds may be isolated from one or more part of the blueberry (e.g., the whole berry, seed, and/or leaf) by physically removing a piece of such fruit, such as by grinding the leaf of the fruit. Such compounds also may be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the berry leaves. According to another embodiment, the anthocyanin-rich extract comprises at least one of delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; and malvidin-6-acetyl-3-glucoside.

According to another embodiment, the anthocyanin-rich extract comprises anthocyanins from maqui berries. According to some such embodiments, the anthocyanin compounds are isolated from maqui berries. According to another embodiment, the compounds are isolated from dried leaves of the maqui berries. According to some such embodiments, the maqui berries are of Aristotelia chilensis. Such compounds may be isolated from one or more part of the maqui berry (e.g., the whole berry, seed, and/or leaf) by physically removing a piece of such fruit, such as by grinding the leaf of the fruit. Such compounds also may be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the berry leaves. According to another embodiment, the anthocyanin-rich extract comprises at least one of delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; and cyanidin 3-glucoside.

The compositions are delivered in therapeutically effective amounts. The term “effective amount” refers to the amount necessary or sufficient to realize a desired biologic effect. Combined with the teachings provided herein, by choosing among the various active compounds and weighing factors such as potency, relative bioavailability, subject body weight, severity of adverse side-effects and preferred mode of administration, an effective prophylactic or therapeutic treatment regimen may be planned which does not cause substantial toxicity and yet is effective to treat the particular subject. The effective amount for any particular application may vary depending on such factors as the disease or condition being treated, the particular flavonoid, anthocyanin, or derivative or variant thereof, including the anthocyanins of, but not limited to, blueberry, such as, for example, delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; malvidin-6-acetyl-3-glucoside (Prior, R, et al. 2001), and the anthocyanins of maqui berries such as, for example, delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; cyanidin 3-glucoside; being administered, the size of the subject, or the severity of the disease or condition. One of ordinary skill in the art may determine empirically the effective amount of a particular inhibitor and/or other therapeutic agent without necessitating undue experimentation. It is preferred generally that a maximum dose be used, that is, the highest safe dose according to some medical judgment. “Dose” and “dosage” are used interchangeably herein.

For any compound described herein the therapeutically effective amount may be determined initially from preliminary in vitro studies and/or animal models. A therapeutically effective dose may also be determined from human data for flavonoids, anthocyanins, or derivatives or variants thereof, for the anthocyanins of blueberry such as, for example, delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; malvidin-6-acetyl-3-glucoside (Prior, R, et al. 2001), and for the anthocyanins of maqui berries such as, for example, delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; cyanidin 3-glucoside, which have been tested in humans, and for compounds which are known to exhibit similar pharmacological activities, such as other related active agents. The applied dose may be adjusted based on the relative bioavailability and potency of the administered compound. Adjusting the dose to achieve maximal efficacy based on the methods described above and other methods as are well-known in the art is well within the capabilities of the ordinarily skilled artisan.

According to another embodiment, the therapeutically effective amount of the anthocyanin-rich extract is from about 10 mg/day to about 10 g/day.

According to another embodiment, the therapeutically effective amount of the anthocyanin-rich extract is from about 0.001 mg/kg body weight to about 10 g/kg body weight. According to some such embodiments, the therapeutically effective amount of the anthocyanin-rich extract is about 0.005 mg/kg body weight. According to some such embodiments, the therapeutically effective amount of the anthocyanin-rich extract is about 0.01 mg/kg body weight. According to some such embodiments, the therapeutically effective amount of the anthocyanin-rich extract is about 0.05 mg/kg body weight. According to some such embodiments, the therapeutically effective amount of the anthocyanin-rich extract is about 0.1 mg/kg body weight. According to some such embodiments, the therapeutically effective amount of the anthocyanin-rich extract is about 1 mg/kg body weight. According to some such embodiments, the therapeutically effective amount of the anthocyanin-rich extract is about 10 mg/kg body weight. According to some such embodiments, the therapeutically effective amount of the anthocyanin-rich extract is about 100 mg/kg body weight. According to some such embodiments, the therapeutically effective amount of the anthocyanin-rich extract is about 250 mg/kg body weight. According to some such embodiments, the therapeutically effective amount of the anthocyanin-rich extract is about 500 mg/kg body weight. According to some such embodiments, the therapeutically effective amount of the anthocyanin-rich extract is about 1 g/kg per body weight. According to some such embodiments, the therapeutically effective amount of the anthocyanin-rich extract is about 2.5 g/kg body weight. According to some such embodiments, the therapeutically effective amount of the anthocyanin-rich extract is about 5 g/kg body weight. According to some such embodiments, the therapeutically effective amount of the anthocyanin-rich extract is about 7.5 g/kg body weight. According to some such embodiments, the therapeutically effective amount of the anthocyanin-rich extract is about 10 g/kg body weight. The formulations of flavonoids, anthocyanins, or derivatives or variants thereof, including, but not limited to, the anthocyanins of blueberry such as, for example, delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; malvidin-6-acetyl-3-glucoside (Prior, R, et al. 2001), and the anthocyanins of maqui berries such as, for example, delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; cyanidin 3-glucoside, may be administered in pharmaceutically acceptable solutions, which routinely may contain pharmaceutically acceptable concentrations of salt, buffering agents, preservatives, compatible carriers, adjuvants, and optionally other therapeutic ingredients.

For use in therapy, an effective amount of the flavonoid, anthocyanin, or derivative or variant thereof, including, but not limited to, the anthocyanins of blueberry such as, for example, delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; malvidin-6-acetyl-3-glucoside (Prior, R, et al. 2001), and the anthocyanins of maqui berries such as, for example, delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; cyanidin 3-glucoside, may be administered to a subject by any mode that delivers the compound to the desired surface. Administering the pharmaceutical composition may be accomplished by any means known to the skilled artisan. The compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally. The flavonoids, anthocyanins, or derivatives or variants thereof, and other therapeutics also may be delivered to a subject during surgery, or other invasive or noninvasive procedure, to treat an underlying condition of a disordered metabolism syndrome.

The flavonoids, anthocyanins, or derivatives or variants thereof, including, but not limited to, the anthocyanins of blueberry such as, for example, delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; malvidin-6-acetyl-3-glucoside (Prior, R, et al. 2001), and the anthocyanins of maqui berries such as, for example, delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; cyanidin 3-glucoside, when it is desirable to deliver them locally, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active compounds may be in powder form for constitution with a suitable vehicle, e.g., sterile pyrogen-free water, before use.

The terms “drug carrier”, “carrier”, or “vehicle” are used interchangeably to refer to carrier materials suitable for administration of the anthocyanin compounds, or variants or derivatives thereof. Carriers and vehicles useful herein include any such materials known in the art which are nontoxic and do not interact with other components. As used herein, the term “pharmaceutically acceptable carrier” refers to any substantially non-toxic carrier conventionally useable for administration in which the compound will remain stable and bioavailable.

According to another embodiment, the pharmaceutically acceptable carrier is an esterified glyceride. In some such embodiments, the pharmaceutically acceptable carrier comprises polyethylene glycol. In some such embodiments, the pharmaceutically acceptable carrier comprises fatty acids. In some such embodiments, the esterified glyceride is saturated. In some such embodiments, the esterified glyceride is unsaturated.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, microencapsulated, and if appropriate, with one or more excipients, encochleated, coated onto microscopic gold particles, contained in liposomes, pellets for implantation into the tissue, or dried onto an object to be rubbed into the tissue. Such pharmaceutical compositions also may be in the form of granules, beads, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, or solubilizers are customarily used as described above. The pharmaceutical compositions are suitable for use in a variety of drug delivery systems. For a brief review of methods for drug delivery, see Langer 1990 Science 249, 1527-1533, which is incorporated herein by reference.

Pharmaceutically acceptable carriers may be esterified glycerides comprising polyethylene glycol or fatty acids. The esterified glycerides may be saturated or unsaturated. Saturated esterified glycerides comprising polyethylene glycol and fatty acids may be obtainable by partial alcoholysis of hydrogenated vegetable oil with polyethylene glycol or by esterification of saturated fatty acids with polyethylene glycol and glycerol. Unsaturated esterified glycerides comprising polyethylene glycol or fatty acids may be obtained by partial alcoholysis of non-hydrogenated vegetable oil with polyethylene glycol. Numerous esterified glycerides comprising polyethylene glycol or fatty acids are known in the art. The esterified glycerides comprising polyethylene glycol or fatty acids may include saturated esterified glycerides with polyethylene glycol and fatty acids consisting of C8-C18 glycerides and polyethylene glycol esters, such as those available under the trade names GELUCIRE®, e.g., GELUCIRE® 33/01, 35/10, 37/02 or 44/14; and LABRAFIL®, e.g., LABRAFIL® WL 2514 CS; unsaturated esterified glycerides with polyethylene glycol and fatty acids consisting of C16-C20 glycerides and polyethylene glycol esters such as those available under the trade name LABRAFIL®, e.g., LABRAFIL® WL 2609 BS, or M 2125 CS; and saturated polyglycolysed C8-C10 glycerides, such as those available under the trade name LABRASOL®

The flavonoids, anthocyanins, or derivatives or variants thereof, and including, but not limited to, the anthocyanins of blueberry such as, for example, delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; malvidin-6-acetyl-3-glucoside (Prior, R, et al. 2001), and the anthocyanins of maqui berries such as, for example, delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; cyanidin 3-glucoside, may be administered per se (neat) or in the form of a pharmaceutically acceptable salt. When used in medicine, the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts may be also obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The formulations may be presented conveniently in unit dosage form and may be prepared by any of the methods well known in the art of pharmacy. All methods include the step of bringing into association a flavonoid, anthocyanin, or derivative or variant thereof, including, but not limited to, the anthocyanins of blueberry such as, for example, delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; malvidin-6-acetyl-3-glucoside (Prior, R, et al. 2001), and the anthocyanins of maqui berries such as, for example, delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; cyanidin 3-glucoside or a pharmaceutically acceptable salt or solvate thereof (“active compound”) with the carrier which constitutes one or more accessory agents. In general, the formulations are prepared by uniformly and intimately bringing into association the active agent with liquid carriers or finely divided solid carriers or both and then, if necessary, shaping the product into the desired formulation.

The pharmaceutical agent or a pharmaceutically acceptable ester, salt, solvate or prodrug thereof may be mixed with other active materials that do not impair the desired action, or with materials that supplement the desired action. Solutions or suspensions used for parenteral, intradermal, subcutaneous, intrathecal, or topical application may include, but are not limited to, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation may be enclosed in ampoules, disposable syringes or multiple dose vials made of glass or plastic. Administered intravenously, particular carriers are physiological saline or phosphate buffered saline (PBS).

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

Injectable depot forms are made by forming microencapsulated matrices of the drug, compound, or active constituent, in biodegradable polymers such as polylactide-polyglycolide. Depending upon the ratio of drug, compound, or active constituent, to polymer and the nature of the particular polymer employed, the rate of drug, compound, or active constituent, release may be controlled. Such long acting formulations may be formulated with suitable polymeric or hydrophobic materials (for example as an emulsion in an acceptable oil) or ion exchange resins, or as sparingly soluble derivatives, for example, as a sparingly soluble salt. Examples of other biodegradable polymers include poly(orthoesters) and poly(anhydrides). Depot injectable formulations are also prepared by entrapping the drug, compound, or active constituent, in liposomes or microemulsions which are compatible with body tissues.

The locally injectable formulations may be sterilized, for example, by filtration through a bacterial-retaining filter or by incorporating sterilizing agents in the form of sterile solid compositions that may be dissolved or dispersed in sterile water or other sterile injectable medium just prior to use. Injectable preparations, for example, sterile injectable aqueous or oleaginous suspensions may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose any bland fixed oil may be employed including synthetic mono- or diglycerides. In addition, fatty acids such as oleic acid are used in the preparation of injectables.

Formulations for parenteral (including but not limited to, subcutaneous, intradermal, intramuscular, intravenous, intrathecal and intraarticular) administration include aqueous and non-aqueous sterile injection solutions that may contain anti-oxidants, buffers, bacteriostats and solutes, which render the formulation isotonic with the blood of the intended recipient; and aqueous and non-aqueous sterile suspensions, which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Another method of formulation of the compositions described herein involves conjugating the compounds described herein to a polymer that enhances aqueous solubility. Examples of suitable polymers include, but are not limited, to polyethylene glycol, poly-(d-glutamic acid), poly-(1-glutamic acid), poly-(1-glutamic acid), poly-(d-aspartic acid), poly-(1-aspartic acid), poly-(1-aspartic acid) and copolymers thereof. For example, polyglutamic acids having molecular weights between about 5,000 to about 100,000, with molecular weights between about 20,000 and about 80,000, and with molecular weights between about 30,000 and about 60,000, may also be used. The polymer may be conjugated via an ester linkage to one or more hydroxyls of an inventive epothilone using a protocol as essentially described by U.S. Pat. No. 5,977,163 which is incorporated herein by reference.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions described within the present invention contain a therapeutically effective amount of flavonoids, anthocyanins, or derivatives or variants thereof, including, but not limited to, the anthocyanins of blueberry such as, for example, delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; malvidin-6-acetyl-3-glucoside (Prior, R, et al. 2001), and/or the anthocyanins of maqui berries such as, for example, delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; cyanidin 3-glucoside, included in a pharmaceutically-acceptable carrier. The therapeutic agent(s), including the flavonoids, anthocyanins, or derivatives or variants thereof, may be provided in particles. The term “particles” as used herein refers to nano or microparticles (or in some instances larger) that may contain in whole or in part the flavonoid, anthocyanin, or derivative or variant thereof, including, but not limited to, the anthocyanins of blueberry such as, for example, delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; malvidin-6-acetyl-3-glucoside, and the anthocyanins of maqui berries such as, for example, delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; cyanidin 3-glucoside. In some embodiments, the particles may contain the therapeutic agent(s) in a core surrounded by a coating. In some embodiments, the therapeutic agent(s) may be dispersed throughout the particles. In some embodiments, the therapeutic agent(s) may be adsorbed into the particles. In some embodiments, the particles may be of any order release kinetics, including zero order release, first order release, second order release, delayed release, sustained release, immediate release, etc., and any combination thereof. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules that contain the flavonoids, anthocyanins, or derivatives or variants thereof, in a solution or in a semi-solid state. The particles may be of virtually any shape.

Both non-biodegradable and biodegradable polymeric materials may be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels as described by Sawhney et al in Macromolecules (1993) 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. In order to prolong the effect of a drug, compound, or active constituent, it often is desirable to slow the absorption of the drug, compound, or active constituent, from subcutaneous, intrathecal, or intramuscular injection. This may be accomplished by the use of a liquid suspension of crystalline or amorphous material with poor water solubility. The rate of absorption of the drug, compound, or active constituent, then depends upon its rate of dissolution which, in turn, may depend upon crystal size and crystalline form. The term “controlled release” is intended to refer to any drug-containing formulation in which the manner and profile of drug, compound, or active constituent, release from the formulation are controlled. This refers to immediate as well as non-immediate release formulations, with non-immediate release formulations including, but not limited to, sustained release and delayed release formulations. The term “sustained release” (also referred to as “extended release”) is used herein in its conventional sense to refer to a drug, compound, or active constituent, formulation that provides for gradual release of a drug, compound, or active constituent, over an extended period of time, and that preferably, although not necessarily, results in substantially constant blood levels of a drug, compound, or active constituent, over an extended time period. Alternatively, delayed absorption of a parenterally administered drug, compound, or active constituent, form is accomplished by dissolving or suspending the drug, compound, or active constituent, in an oil vehicle. The term “delayed release” is used herein in its conventional sense to refer to a drug, compound, or active constituent, formulation in which there is a time delay between administration of the formulation and the release of the drug, compound, or active constituent, there from. “Delayed release” may or may not involve gradual release of drug, compound, or active constituent, over an extended period of time, and thus may or may not be “sustained release.”

Use of a long-term sustained release implant may be particularly suitable for treatment of chronic conditions. The term “long-term” release, as used herein, means that the implant is constructed and arranged to deliver therapeutic levels of the active ingredient for at least 7 days, and preferably about 30 to about 60 days. Long-term sustained release implants are well-known to those of ordinary skill in the art and include some of the release systems described above.

The term “diluent” as used herein refers to an agent used for making thinner or less concentrated by admixture. Diluents typically are inactive ingredients. Diluents include, for example, but not limited to, water, starch, cellulose derivatives, and lubricants, such as magnesium stearate. A diluent may be referred to as a “diluting agent.” The terms “dilute” or “diluting” as used herein refers to weaken, temper, mitigate, diminish, reduce in strength, force, or efficiency of by admixture.

According to another embodiment, the present invention provides a beverage formulation comprising a pharmaceutical composition containing a therapeutically effective amount of an anthocyanin-rich extract derived from blueberries for treating a disordered metabolism syndrome; a beverage component; and a diluent. In some embodiments, the present invention provides a beverage formulation comprising a therapeutically effective amount of an anthocyanin-rich extract derived from blueberries, a beverage component; and a diluent wherein the anthocyanin-rich extract derived from blueberries is in an amount effective to treat a patient suffering from diabetes. In some embodiments, the present invention provides a beverage formulation comprising a therapeutically effective amount of an anthocyanin-rich extract derived from blueberries, a beverage component, and a diluent wherein the anthocyanin-rich extract derived from blueberries is in an amount effective to treat a patient suffering from metabolic syndrome.

According to another embodiment, the present invention provides a beverage formulation comprising a pharmaceutical composition containing a therapeutically effective amount of an anthocyanin-rich extract derived from maqui berries for treating a disordered metabolism syndrome; a beverage component; and a diluent. In some embodiments, the present invention provides a beverage formulation comprising a therapeutically effective amount of an anthocyanin-rich extract derived from maqui berries, a beverage component; and a diluent wherein the anthocyanin-rich extract derived from maqui berries is in an amount effective to treat a patient suffering from diabetes. In some embodiments, the present invention provides a beverage formulation comprising a therapeutically effective amount of an anthocyanin-rich extract derived from maqui berries, a beverage component, and a diluent wherein the anthocyanin-rich extract derived from maqui berries is in an amount effective to treat a patient suffering from metabolic syndrome.

The term “beverage” as used herein refers to a solution ingested in a liquid form. A solution generally is considered as a homogeneous mixture of two or more substances; it is frequently, though not necessarily, a liquid. In a solution, the molecules of the solute (or dissolved substance) are uniformly distributed among those of the solvent. A suspension is a dispersion (mixture) in which a finely-divided species is combined with another species, with the former being so finely divided and mixed that it doesn't rapidly settle out. In everyday life, the most common suspensions are those of solids in liquid. Among the acceptable vehicles and solvents that may be employed are water, Ringer's solution, and isotonic sodium chloride solution. The term “component” as used herein refers to a constituent part, element or ingredient.

According to another embodiment, the beverage component further comprises other components to enhance the efficacy of the beverage in providing benefits such as fighting infection, providing a desirable nutritional profile, and/or providing enhanced organoleptic properties. In some such embodiments, the beverage component is tea. In some such embodiments, the beverage component is a carbonated drink. In some such embodiments, the beverage component is a nutritional drink. In some such embodiments, the beverage component is a frozen drink. In some such embodiments, the beverage component is a cola. In some such embodiments, the cola profile features carbonated water. In some such embodiments, the cola profile features aspartame. In some such embodiments, the cola profile features a sugar substitute. In some such embodiments, the cola profile features acesulfame potassium. In some such embodiments, the cola profile features an acid component. In some such embodiments, the cola profile features caffeine. The beverage component also may be other beverage components as known in the art.

According to another embodiment, the beverage component further comprises one or more bracers. In another embodiment, the beverage component further comprises flavanols. In another embodiment, the beverage component further comprises non-caloric sweeteners. In another embodiment, the beverage component further comprises vitamins. In another embodiment, the beverage component further comprises emulsions. In another embodiment, the beverage component further comprises flavoring agents. In another embodiment, the beverage component further comprises coloring agents. In another embodiment, the beverage component further comprises preservatives. In another embodiment, the beverage component further comprises acidulants. In another embodiment, the beverage component further comprises a diluent. In another embodiment, the beverage component further comprises water. In another embodiment, the beverage component further comprises carbonation components.

In some such embodiments, the components may be dispersed, solubilized, or otherwise mixed into the beverage formulation of the instant invention.

### Methods for Treating Disordered Metabolism Syndromes

According to another aspect, the present invention provides a method for treating a disordered metabolism syndrome. In some embodiments, the method comprises the steps: (a) administering a therapeutically effective amount of a composition to a subject in need thereof, wherein the composition comprises an anthocyanin-rich extract derived from berries and a pharmaceutically acceptable carrier; thereby treating the disordered metabolism syndrome.

The term “food” as used herein refers to articles used for food or drink for man or other animals, and articles used for components of any such article.

The term “health” or “healthy” as used herein refers to a general condition of the body or mind with references to soundness and vigor, as well as freedom from disease or ailment.

The term “microbe” or “microoranism” are used interchangeably herein to refer to an organism too small to be seen clearly with the naked eye, including, but not limited to, microscopic bacteria, fungi (molds), algae, protozoa and viruses.

The term “microbial raw material” as used herein refers to a fresh or processed (for example, concentrated, frozen, dried, dissolved, liquefied, pelleted) part of a microbial culture.

The term “microbial ingredient” as used herein refers to a component that originates from a microbial raw material.

The term “microbial product” as used herein refers to a finished, labeled product that contains matter derived from a microbial culture.

The terms “soluble” and “solubility” refer to the property of being susceptible to being dissolved in a specified fluid (solvent). The term “insoluble” refers to the property of a material that has minimal or limited solubility in a specified solvent.

The term “well-being” as used herein refers to a subject's physical and mental soundness.

The term “botanical raw material” as used herein refers to a fresh or processed (for example, cleaned, frozen, dried, sliced, dissolved, or liquefied) part of a single species of plant or a fresh or processed alga or microscopic fungus.

The term “botanical ingredient” as used herein refers to a component that originates from a botanical raw material.

The term “botanical product” as used herein refers to a finished, labeled product that contains vegetable matter, which may include plant materials, algae, macroscopic fungi, or combinations thereof. Depending in part on its intended use, a botanical product may be a food, drug, medical device or cosmetic.

The term “botanical extract” as used herein refers to a product prepared by separating, by chemical or physical process, medicinally active portions of a plant from the inactive or inert components. The botanical extracts prepared according to the present invention may be obtained by means of a solvent, optionally under pressure and/or heat. Botanical extracts are often complex, necessitating the use of high resolution analytical techniques such as HPLC and LC/MS.

The term “comestible” as used herein refers to a material that is suitable for human consumption, including a material that can be ingested by oral and by a non-oral means, for example, an inhalant or snuff.

The term “nutraceutical” as used herein refers to a food or naturally occurring food supplement thought to have a beneficial effect on human health or well-being. Nutraceutical may also be referred to as botanical supplement, ergogenic aid, functional food, herbal, medical food, or nutriceutical.

The terms “dietary supplement” and “nutritional supplement” are used interchangeably herein to mean (1) a product intended to supplement the diet that bears or contains one or more of the following dietary ingredients: [A] a vitamin, [B] a mineral, [C] a herb or other botanical, [D] an amino acid, [E] a dietary substance for sue by man to supplement the diet by increasing the total dietary intake, or [F] a concentrate, metabolite, constituent, extract, or combination of any ingredient described in classes [A], [B]. [C], [D], or [E]; and (2) a product that (A)(i) is intended for ingestion; (B) is not represented for use as a conventional food or as a sole item of a meal or the diet; and (C) is labeled as a dietary supplement.

The term “essentially free” as used herein means less than about 10% of the amount of found in unprocessed material. For example, if a blueberry contains about 1% w/w anthocyanin, then an extract that is essentially free of anthocyanin would contain less than about 0.1% anthocyanin w/w (excluding additional mass due to dilution in water).

The term “active constituent” as used herein refers to the chemical constituent in a botanical raw material or a microbial raw material that is responsible for the intended therapeutic effect.

According to one embodiment, the disordered metabolism syndrome is diabetes. According to another embodiment, the disordered metabolism syndrome is diabetes mellitus. According to another embodiment, the disordered metabolism syndrome is Type 1 diabetes mellitus. According to another embodiment, the disordered metabolism syndrome is Type 2 diabetes mellitus. According to another embodiment, the disordered metabolism syndrome is juvenile diabetes. According to another embodiment, the disordered metabolism syndrome is gestational diabetes. According to another embodiment, the disordered metabolism syndrome is metabolic syndrome. According to another embodiment, the disordered metabolism syndrome is metabolic syndrome X. According to another embodiment, the disordered metabolism syndrome is syndrome X. According to another embodiment, the disordered metabolism syndrome is insulin resistance syndrome. According to another embodiment, the disordered metabolism syndrome is Reaven's syndrome. According to another embodiment, the disordered metabolism syndrome is CHAOS.

According to another embodiment, the composition is a pharmaceutical composition. According to another embodiment, the composition is a nutraceutical composition. According to another embodiment, the composition is a dietary supplement. According to another embodiment, the composition is a botanical product. According to another embodiment, the composition is a botanical extract. According to another embodiment, the composition is a comestible.

According to another embodiment, the berries are blueberries. In some such embodiments, the blueberries are wild lowbush blueberries. In some such embodiments, the blueberries are of Vaccinium angustifolium A. According another embodiment, the berries are maqui berries. In some such embodiments, the berries are of Aristotelia chilensis. According to another embodiment, the blueberry is a fruit of the plant genus Vaccinium. According to another embodiment, the maqui berry is a fruit of the plant genus Aristotelia. According to another embodiment, the berry is an elderberry from the plant genus Sambucus. According to another embodiment, the berry is a chokeberry from the plant genus Aronia. According to another embodiment, the berry is a blackcurrant from the plant genus Ribes. According to another embodiment, the berry is a blackcurrant from the plant Ribes nigrum.

According to another embodiment, the anthocyanin-rich extract comprises anthocyanins from blueberries. According to some such embodiments, the compounds are isolated from blueberries. According to another embodiment, the compounds are isolated from dried leaves of the blueberry. According to some such embodiments, the blueberries are wild lowbush blueberries. According to some such embodiments, the blueberries are of Vaccinium angustifolium A. Such compounds may be isolated from one or more part of the blueberry (e.g., the whole berry, seed, and/or leaf) by physically removing a piece of such fruit, such as by grinding the leaf of the fruit. Such compounds also may be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the leaves of berries. According to another embodiment, the anthocyanin-rich extract comprises at least one of delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; and malvidin-6-acetyl-3-glucoside.

According to another embodiment, the anthocyanin-rich extract comprises anthocyanins from maqui berries. According to some such embodiments, the compounds are isolated from maqui berries. According to another embodiment, the compounds are isolated from dried leaves of the maqui berries. According to some such embodiments, the maqui berries are of Aristotelia chilensis. Such compounds may be isolated from one or more part of the maqui berry (e.g., the whole berry, seed, and/or leaf) by physically removing a piece of such fruit, such as grinding the leaf of the fruit. Such compounds also may be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the leaves of berries. According to another embodiment, the anthocyanin-rich extract comprises at least one of delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; and cyanidin 3-glucoside.

According to another embodiment, the therapeutically effective amount is from about 0.001 mg/kg body weight to about 10 g/kg body weight.

According to another embodiment, the therapeutically effective amount of the anthocyanin-rich extract is from about 0.001 mg/kg body weight to about 10 g/kg body weight. According to some such embodiments, the therapeutically effective amount of the anthocyanin-rich extract is about 0.005 mg/kg body weight. According to some such embodiments, the therapeutically effective amount of the anthocyanin-rich extract is about 0.01 mg/kg body weight. According to some such embodiments, the therapeutically effective amount of the anthocyanin-rich extract is about 0.05 mg/kg body weight. According to some such embodiments, the therapeutically effective amount of the anthocyanin-rich extract is about 0.1 mg/kg body weight. According to some such embodiments, the therapeutically effective amount of the anthocyanin-rich extract is about 1 mg/kg body weight. According to some such embodiments, the therapeutically effective amount of the anthocyanin-rich extract is about 10 mg/kg body weight. According to some such embodiments, the therapeutically effective amount of the anthocyanin-rich extract is about 100 mg/kg body weight. According to some such embodiments, the therapeutically effective amount of the anthocyanin-rich extract is about 250 mg/kg body weight. According to some such embodiments, the therapeutically effective amount of the anthocyanin-rich extract is about 500 mg/kg body weight. According to some such embodiments, the therapeutically effective amount of the anthocyanin-rich extract is about 1 g/kg per body weight. According to some such embodiments, the therapeutically effective amount of the anthocyanin-rich extract is about 2.5 g/kg body weight. According to some such embodiments, the therapeutically effective amount of the anthocyanin-rich extract is about 5 g/kg body weight. According to some such embodiments, the therapeutically effective amount of the anthocyanin-rich extract is about 7.5 g/kg body weight. According to some such embodiments, the therapeutically effective amount of the anthocyanin-rich extract is about 10 g/kg body weight.

According to some such embodiments, wherein the disordered metabolism syndrome is diabetes, the method lowers the levels of blood sugar in a subject. According to some such embodiments, wherein the disordered metabolism syndrome is diabetes, the method increases insulin sensitivity. According to some such embodiments, wherein the disordered metabolism syndrome is metabolic syndrome, the method lowers the level of blood sugar in a subject. According to some such embodiments, wherein the disordered metabolism syndrome is metabolic syndrome, the method increases insulin sensitivity.

According to another embodiment, the hyperglycemia symptom is decreased by about 5% relative to a healthy subject. According to another embodiment, the hyperglycemia symptom is decreased by about 10% relative to a healthy subject. According to another embodiment, the hyperglycemia symptom is decreased by about 15% relative to a healthy subject. According to another embodiment, the hyperglycemia symptom is decreased by about 20% relative to a healthy subject. According to another embodiment, the hyperglycemia symptom is decreased by about 25% relative to a healthy subject. According to another embodiment, the hyperglycemia symptom is decreased by about 30% relative to a healthy subject. According to another embodiment, the hyperglycemia symptom is decreased by about 35% relative to a healthy subject. According to another embodiment, the hyperglycemia symptom is decreased by about 40% relative to a healthy subject. According to another embodiment, the hyperglycemia symptom is decreased by about 45% relative to a healthy subject. According to another embodiment, the hyperglycemia symptom is decreased by about 50% relative to a healthy subject. According to another embodiment, the hyperglycemia symptom is decreased by about 55% relative to a healthy subject. According to another embodiment, the hyperglycemia symptom is decreased by about 60% relative to a healthy subject. According to another embodiment, the hyperglycemia symptom is decreased by about 65% relative to a healthy subject. According to another embodiment, the hyperglycemia symptom is decreased by about 70% relative to a healthy subject. According to another embodiment, the hyperglycemia symptom is decreased by about 75% relative to a healthy subject. According to another embodiment, the hyperglycemia symptom is decreased by about 80% relative to a healthy subject. According to another embodiment, the hyperglycemia symptom is decreased by about 85% relative to a healthy subject. According to another embodiment, the hyperglycemia symptom is decreased by about 90% relative to a healthy subject. According to another embodiment, the hyperglycemia symptom is decreased by about 95% relative to a healthy subject.

According to another aspect, the present invention provides a method for reducing symptoms of a disordered metabolism syndrome, the method comprising the steps: (a) administering a therapeutically effective amount of a composition to a subject in need thereof; wherein the pharmaceutical composition comprises an anthocyanin-rich extract derived from berries and a pharmaceutically acceptable carrier; thereby alleviating the symptoms of the disordered metabolism syndrome.

According to one embodiment, the composition is a pharmaceutical composition. According to another embodiment, the composition is a nutraceutical composition. According to another embodiment, the composition is a dietary supplement. According to another embodiment, the composition is a botanical product. According to another embodiment, the composition is a botanical extract. According to another embodiment, the composition is a comestible.

According to one embodiment, the disordered metabolism syndrome is diabetes. According to another embodiment, the disordered metabolism syndrome is diabetes mellitus. According to another embodiment, the disordered metabolism syndrome is Type 1 diabetes mellitus. According to another embodiment, the disordered metabolism syndrome is Type 2 diabetes mellitus. According to another embodiment, the disordered metabolism syndrome is juvenile diabetes. According to another embodiment, the disordered metabolism syndrome is gestational diabetes. According to another embodiment, the disordered metabolism syndrome is metabolic syndrome. According to another embodiment, the disordered metabolism syndrome is metabolic syndrome X. According to another embodiment, the disordered metabolism syndrome is syndrome X. According to another embodiment, the disordered metabolism syndrome is insulin resistance syndrome. According to another embodiment, the disordered metabolism syndrome is Reaven's syndrome. According to another embodiment, the disordered metabolism syndrome is CHAOS.

According to one embodiment, symptoms of diabetes that may be treated according to the invention include, but are not limited to, abnormal wound healing, symptoms of a heart attack, symptoms of a stroke, symptoms of peripheral vascular disease, symptoms of kidney disease, kidney failure, blindness, neuropathy, inflammation, impotence, arteriosclerosis, diabetic retinopathy (possibly leading to blindness), cataracts, nephropathy, increased risk of infections, hypertension, nerve disease, risk of amputations, diabetic ketoacidosis, and dementia.

According to another embodiment, symptoms of metabolic syndrome that may be treated according to the invention include, but are not limited to, fasting hypoglycemia, high blood pressure, decreased high-density lipoprotein cholesterol, elevated triglycerides and elevated uric acid levels.

According to another embodiment, the symptoms of the disordered metabolism syndrome is at least one of elevated blood pressure, elevated levels of high-density lipoprotein cholesterol, elevated levels of triglycerides, and elevated levels of fasting plasma glucose.

According to another embodiment, the berries are blueberries. In some such embodiments, the blueberries are wild lowbush blueberries. In some such embodiments, the blueberries are of Vaccinium angustifolium A. In another embodiment, the berries are maqui berries. In some such embodiments, the berries are of Aristotelia chilensis. According to another embodiment, the blueberry is a fruit of the plant genus Vaccinium. According to another embodiment, the maqui berry is a fruit of the plant genus Aristotelia. According to another embodiment, the berry is an elderberry from the plant genus Sambucus. According to another embodiment, the berry is a chokeberry from the plant genus Aronia. According to another embodiment, the berry is a blackcurrant from the plant genus Ribes. According to another embodiment, the berry is a blackcurrant from the plant Ribes nigrum.

According to another embodiment, the anthocyanin-rich extract comprises anthocyanins from blueberries. According to some such embodiments, the compounds are isolated from blueberries. According to another embodiment, the compounds are isolated from dried leaves of the blueberry. According to some such embodiments, the blueberries are wild lowbush blueberries. According to some such embodiments, the blueberries are of Vaccinium angustifolium A. Such compounds may be isolated from one or more part of the blueberry (e.g., the whole berry, seed, and/or leaf) by physically removing a piece of such fruit, such as by grinding the leaf of the fruit. Such compounds may also be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the leaves. According to another embodiment, the anthocyanin-rich extract comprises at least one of delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; and malvidin-6-acetyl-3-glucoside.

According to another embodiment, the anthocyanin-rich extract comprises anthocyanins from maqui berries. According to some such embodiments, the compounds are isolated from maqui berries. According to another embodiment, the compounds are isolated from dried leaves of the maqui berries. According to some such embodiments, the maqui berries are of Aristotelia chilensis. Such compounds may be isolated from one or more part of the maqui berry (e.g., the whole berry, seed, and/or leaf) by physically removing a piece of such fruit, such as grinding the leaf of the fruit. Such compounds also may be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the leaves. According to another embodiment, the anthocyanin-rich extract comprises at least one of delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; and cyanidin 3-glucoside.

According to another embodiment, the therapeutically effective amount is from about 0.001 mg/kg body weight to about 10 g/kg body weight.

According to another embodiment, the therapeutically effective amount of the anthocyanin-rich extract is from about 0.001 mg/kg body weight to about 10 g/kg body weight. According to some such embodiments, the therapeutically effective amount of the anthocyanin-rich extract is about 0.005 mg/kg body weight. According to some such embodiments, the therapeutically effective amount of the anthocyanin-rich extract is about 0.01 mg/kg body weight. According to some such embodiments, the therapeutically effective amount of the anthocyanin-rich extract is about 0.05 mg/kg body weight. According to some such embodiments, the therapeutically effective amount of the anthocyanin-rich extract is about 0.1 mg/kg body weight. According to some such embodiments, the therapeutically effective amount of the anthocyanin-rich extract is about 1 mg/kg body weight. According to some such embodiments, the therapeutically effective amount of the anthocyanin-rich extract is about 10 mg/kg body weight. According to some such embodiments, the therapeutically effective amount of the anthocyanin-rich extract is about 100 mg/kg body weight. According to some such embodiments, the therapeutically effective amount of the anthocyanin-rich extract is about 250 mg/kg body weight. According to some such embodiments, the therapeutically effective amount of the anthocyanin-rich extract is about 500 mg/kg body weight. According to some such embodiments, the therapeutically effective amount of the anthocyanin-rich extract is about 1 g/kg per body weight. According to some such embodiments, the therapeutically effective amount of the anthocyanin-rich extract is about 2.5 g/kg body weight. According to some such embodiments, the therapeutically effective amount of the anthocyanin-rich extract is about 5 g/kg body weight. According to some such embodiments, the therapeutically effective amount of the anthocyanin-rich extract is about 7.5 g/kg body weight. According to some such embodiments, the therapeutically effective amount of the anthocyanin-rich extract is about 10 g/kg body weight.

According to another aspect, the present invention provides a method for treating hyperglycemia associated with a disordered metabolism syndrome, the method comprising the steps: (a) administering a therapeutically effective amount of a composition to a subject in need thereof; wherein the composition comprises an anthocyanin-rich extract derived from berries and a pharmaceutically acceptable carrier; thereby lowering blood glucose levels.

The term “hyperglycemia” as used herein refers to an abnormally high level of glucose in the blood. The term “hypoglycemia” as used herein refers to an abnormally low level of glucose in the blood.

The term “hypertension” as used herein refers to high blood pressure. Generally, high blood is a blood pressure reading of 140/90 mmHg (millimeters of mercury) or higher.

According to one embodiment, the composition is a pharmaceutical composition. According to another embodiment, the composition is a nutraceutical composition. According to another embodiment, the composition is a dietary supplement. According to another embodiment, the composition is a botanical product. According to another embodiment, the composition is a botanical extract. According to another embodiment, the composition is a comestible.

According to another embodiment, the berries are blueberries. In some such embodiments, the blueberries are wild lowbush blueberries. In some such embodiments, the blueberries are of Vaccinium angustifolium A. According to another embodiment, the berries are maqui berries. In some such embodiments, the berries are of Aristotelia chilensis. According to another embodiment, the blueberry is a fruit of the plant genus Vaccinium. According to another embodiment, the maqui berry is a fruit of the plant genus Aristotelia. According to another embodiment, the berry is an elderberry from the plant genus Sambucus. According to another embodiment, the berry is a chokeberry from the plant genus Aronia. According to another embodiment, the berry is a blackcurrant from the plant genus Ribes. According to another embodiment, the berry is a blackcurrant from the plant Ribes nigrum.

According to another embodiment, the anthocyanin-rich extract comprises anthocyanins from blueberries. According to some such embodiments, the compounds are isolated from blueberries. According to another embodiment, the compounds are isolated from dried leaves of the blueberry. According to some such embodiments, the blueberries are wild lowbush blueberries. According to some such embodiments, the blueberries are of Vaccinium angustifolium A. Such compounds may be isolated from one or more part of the blueberry (e.g., the whole berry, seed, and/or leaf) by physically removing a piece of such fruit, such as by grinding the leaf of the fruit. Such compounds also may be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the leaves of berries. According to another embodiment, the anthocyanin-rich extract comprises at least one of delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; and malvidin-6-acetyl-3-glucoside.

According to another embodiment, the anthocyanin-rich extract comprises anthocyanins from maqui berries. According to some such embodiments, the compounds are isolated from maqui berries. According to another embodiment, the compounds are isolated from dried leaves of the maqui berries. According to some such embodiments, the maqui berries are of Aristotelia chilensis. Such compounds may be isolated from one or more part of the maqui berry (e.g., the whole berry, seed, and/or leaf) by physically removing a piece of such fruit, such as grinding the leaf of the fruit. Such compounds also may be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the leaves of berries. According to another embodiment, the anthocyanin-rich extract comprises at least one of delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; and cyanidin 3-glucoside.

According to another embodiment, the disordered metabolism syndrome is diabetes. According to another embodiment, the disordered metabolism syndrome is diabetes mellitus. According to another embodiment, the disordered metabolism syndrome is Type 1 diabetes mellitus. According to another embodiment, the disordered metabolism syndrome is Type 2 diabetes mellitus. According to another embodiment, the disordered metabolism syndrome is juvenile diabetes. According to another embodiment, the disordered metabolism syndrome is gestational diabetes. According to another embodiment, the disordered metabolism syndrome is metabolic syndrome. According to another embodiment, the disordered metabolism syndrome is metabolic syndrome X. According to another embodiment, the disordered metabolism syndrome is syndrome X. According to another embodiment, the disordered metabolism syndrome is insulin resistance syndrome. According to another embodiment, the disordered metabolism syndrome is Reaven's syndrome. According to another embodiment, the disordered metabolism syndrome is CHAOS.

According to another embodiment, the therapeutically effective amount is from about 0.001 mg/kg body weight to about 10 g/kg body weight.

According to another embodiment, the therapeutically effective amount of the anthocyanin-rich extract is from about 0.001 mg/kg body weight to about 10 g/kg body weight. According to some such embodiments, the therapeutically effective amount of the anthocyanin-rich extract is about 0.005 mg/kg body weight. According to some such embodiments, the therapeutically effective amount of the anthocyanin-rich extract is about 0.01 mg/kg body weight. According to some such embodiments, the therapeutically effective amount of the anthocyanin-rich extract is about 0.05 mg/kg body weight. According to some such embodiments, the therapeutically effective amount of the anthocyanin-rich extract is about 0.1 mg/kg body weight. According to some such embodiments, the therapeutically effective amount of the anthocyanin-rich extract is about 1 mg/kg body weight. According to some such embodiments, the therapeutically effective amount of the anthocyanin-rich extract is about 10 mg/kg body weight. According to some such embodiments, the therapeutically effective amount of the anthocyanin-rich extract is about 100 mg/kg body weight. According to some such embodiments, the therapeutically effective amount of the anthocyanin-rich extract is about 250 mg/kg body weight. According to some such embodiments, the therapeutically effective amount of the anthocyanin-rich extract is about 500 mg/kg body weight. According to some such embodiments, the therapeutically effective amount of the anthocyanin-rich extract is about 1 g/kg per body weight. According to some such embodiments, the therapeutically effective amount of the anthocyanin-rich extract is about 2.5 g/kg body weight. According to some such embodiments, the therapeutically effective amount of the anthocyanin-rich extract is about 5 g/kg body weight. According to some such embodiments, the therapeutically effective amount of the anthocyanin-rich extract is about 7.5 g/kg body weight. According to some such embodiments, the therapeutically effective amount of the anthocyanin-rich extract is about 10 g/kg body weight.

According to another aspect, the present invention provides a method for alleviating symptoms of diabetes in a subject, the method comprising the steps: (a) administering a therapeutically effective amount of a pharmaceutical composition to a subject in need thereof; wherein the pharmaceutical composition comprises an anthocyanin-rich extract derived from berries and a pharmaceutically acceptable carrier; thereby reducing the symptoms of diabetes.

According to one embodiment, the berries are blueberries. In some such embodiments, the blueberries are wild lowbush blueberries. In some such embodiments, the blueberries are of Vaccinium angustifolium A. According to another embodiment, the berries are maqui berries. In some such embodiments, the berries are of Aristotelia chilensis. According to another embodiment, the blueberry is a fruit of the plant genus Vaccinium. According to another embodiment, the maqui berry is a fruit of the plant genus Aristotelia. According to another embodiment, the berry is an elderberry from the plant genus Sambucus. According to another embodiment, the berry is a chokeberry from the plant genus Aronia. According to another embodiment, the berry is a blackcurrant from the plant genus Ribes. According to another embodiment, the berry is a blackcurrant from the plant Ribes nigrum.

According to another embodiment, the anthocyanin-rich extract comprises anthocyanins from blueberries. According to some such embodiments, the compounds are isolated from blueberries. According to another embodiment, the compounds are isolated from dried leaves of the blueberry. According to some such embodiments, the blueberries are wild lowbush blueberries. According to some such embodiments, the blueberries are of Vaccinium angustifolium A. Such compounds may be isolated from one or more part of the blueberry (e.g., the whole berry, seed, and/or leaf) by physically removing a piece of such fruit, such as by grinding the leaf of the fruit. Such compounds also may be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the leaves of berries. According to another embodiment, the anthocyanin-rich extract comprises at least one of delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; and malvidin-6-acetyl-3-glucoside.

According to another embodiment, the anthocyanin-rich extract comprises anthocyanins from maqui berries. According to some such embodiments, the compounds are isolated from maqui berries. According to another embodiment, the compounds are isolated from dried leaves of the maqui berries. According to some such embodiments, the maqui berries are of Aristotelia chilensis. Such compounds may be isolated from one or more part of the maqui berry (e.g., the whole berry, seed, and/or leaf) by physically removing a piece of such fruit, such as grinding the leaf of the fruit. Such compounds also may be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the leaves of berries. According to another embodiment, the anthocyanin-rich extract comprises at least one of delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; and cyanidin 3-glucoside.

According to another embodiment, the therapeutically effective amount is from about 0.001 mg/kg body weight to about 10 g/kg body weight.

According to another embodiment, the therapeutically effective amount of the anthocyanin-rich extract is from about 0.001 mg/kg body weight to about 10 g/kg body weight. According to some such embodiments, the therapeutically effective amount of the anthocyanin-rich extract is about 0.005 mg/kg body weight. According to some such embodiments, the therapeutically effective amount of the anthocyanin-rich extract is about 0.01 mg/kg body weight. According to some such embodiments, the therapeutically effective amount of the anthocyanin-rich extract is about 0.05 mg/kg body weight. According to some such embodiments, the therapeutically effective amount of the anthocyanin-rich extract is about 0.1 mg/kg body weight. According to some such embodiments, the therapeutically effective amount of the anthocyanin-rich extract is about 1 mg/kg body weight. According to some such embodiments, the therapeutically effective amount of the anthocyanin-rich extract is about 10 mg/kg body weight. According to some such embodiments, the therapeutically effective amount of the anthocyanin-rich extract is about 100 mg/kg body weight. According to some such embodiments, the therapeutically effective amount of the anthocyanin-rich extract is about 250 mg/kg body weight. According to some such embodiments, the therapeutically effective amount of the anthocyanin-rich extract is about 500 mg/kg body weight. According to some such embodiments, the therapeutically effective amount of the anthocyanin-rich extract is about 1 g/kg per body weight. According to some such embodiments, the therapeutically effective amount of the anthocyanin-rich extract is about 2.5 g/kg body weight. According to some such embodiments, the therapeutically effective amount of the anthocyanin-rich extract is about 5 g/kg body weight. According to some such embodiments, the therapeutically effective amount of the anthocyanin-rich extract is about 7.5 g/kg body weight. According to some such embodiments, the therapeutically effective amount of the anthocyanin-rich extract is about 10 g/kg body weight.

According to another embodiment, symptoms of diabetes that may be treated according to the invention include, but are not limited to, abnormal wound healing, symptoms of a heart attack, symptoms of a stroke, symptoms of peripheral vascular disease, symptoms of kidney disease, kidney failure, blindness, neuropathy, inflammation, impotence, arteriosclerosis, diabetic retinopathy (possibly leading to blindness), cataracts, nephropathy, increased risk of infections, hypertension, nerve disease, risk of amputations, diabetic ketoacidosis, and dementia.

According to another aspect, the present invention provides a method for alleviating symptoms of metabolic syndrome in a subject, the method comprising the steps: (a) administering a therapeutically effective amount of a composition to a subject in need thereof; wherein the composition comprises an anthocyanin-rich extract derived from berries and a pharmaceutically acceptable carrier; thereby reducing the symptom of insulin resistance.

According to one embodiment, the composition is a pharmaceutical composition. According to another embodiment, the composition is a nutraceutical composition. According to another embodiment, the composition is a dietary supplement. According to another embodiment, the composition is a botanical product. According to another embodiment, the composition is a botanical extract. According to another embodiment, the composition is a comestible.

According to another embodiment, the berries are blueberries. In some such embodiments, the blueberries are wild lowbush blueberries. In some such embodiments, the blueberries are of Vaccinium angustifolium A. According to another embodiment, the berries are maqui berries. In some such embodiments, the berries are of Aristotelia chilensis. According to another embodiment, the blueberry is a fruit of the plant genus Vaccinium. According to another embodiment, the maqui berry is a fruit of the plant genus Aristotelia. According to another embodiment, the berry is an elderberry from the plant genus Sambucus. According to another embodiment, the berry is a chokeberry from the plant genus Aronia. According to another embodiment, the berry is a blackcurrant from the plant genus Ribes. According to another embodiment, the berry is a blackcurrant from the plant Ribes nigrum.

According to another embodiment, the anthocyanin-rich extract comprises anthocyanins from blueberries. According to some such embodiments, the compounds are isolated from blueberries. According to another embodiment, the compounds are isolated from dried leaves of the blueberry. According to some such embodiments, the blueberries are wild lowbush blueberries. According to some such embodiments, the blueberries are of Vaccinium angustifolium A. Such compounds may be isolated from one or more part of the blueberry (e.g., the whole berry, seed, and/or leaf) by physically removing a piece of such fruit, such as by grinding the leaf of the fruit. Such compounds also may be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the leaves of berries. According to another embodiment, the anthocyanin-rich extract comprises at least one of delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; and malvidin-6-acetyl-3-glucoside.

According to another embodiment, the anthocyanin-rich extract comprises anthocyanins from maqui berries. According to some such embodiments, the compounds are isolated from maqui berries. According to another embodiment, the compounds are isolated from dried leaves of the maqui berries. According to some such embodiments, the maqui berries are of Aristotelia chilensis. Such compounds may be isolated from one or more part of the maqui berry (e.g., the whole berry, seed, and/or leaf) by physically removing a piece of such fruit, such as by grinding the leaf of the fruit. Such compounds also may be isolated from the berry by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). According to another embodiment, the anthocyanin-rich extract contains only hydrophilic compounds (e.g., isolated by using a hydrophilic solvent, such as water or ethanol). According to another embodiment, the anthocyanin-rich extract is an aqueous extract prepared from the berries. According to another embodiment, the anthocyanin-rich extract is an aqueous extract prepared from the leaves of berries. According to another embodiment, the anthocyanin-rich extract comprises at least one of delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; and cyanidin 3-glucoside.

According to another embodiment, the therapeutically effective amount is from about 0.001 mg/kg body weight to about 10 g/kg body weight.

According to another embodiment, the therapeutically effective amount of the anthocyanin-rich extract is from about 0.001 mg/kg body weight to about 10 g/kg body weight. According to some such embodiments, the therapeutically effective amount of the anthocyanin-rich extract is about 0.005 mg/kg body weight. According to some such embodiments, the therapeutically effective amount of the anthocyanin-rich extract is about 0.01 mg/kg body weight. According to some such embodiments, the therapeutically effective amount of the anthocyanin-rich extract is about 0.05 mg/kg body weight. According to some such embodiments, the therapeutically effective amount of the anthocyanin-rich extract is about 0.1 mg/kg body weight. According to some such embodiments, the therapeutically effective amount of the anthocyanin-rich extract is about 1 mg/kg body weight. According to some such embodiments, the therapeutically effective amount of the anthocyanin-rich extract is about 10 mg/kg body weight. According to some such embodiments, the therapeutically effective amount of the anthocyanin-rich extract is about 100 mg/kg body weight. According to some such embodiments, the therapeutically effective amount of the anthocyanin-rich extract is about 250 mg/kg body weight. According to some such embodiments, the therapeutically effective amount of the anthocyanin-rich extract is about 500 mg/kg body weight. According to some such embodiments, the therapeutically effective amount of the anthocyanin-rich extract is about 1 g/kg per body weight. According to some such embodiments, the therapeutically effective amount of the anthocyanin-rich extract is about 2.5 g/kg body weight. According to some such embodiments, the therapeutically effective amount of the anthocyanin-rich extract is about 5 g/kg body weight. According to some such embodiments, the therapeutically effective amount of the anthocyanin-rich extract is about 7.5 g/kg body weight. According to some such embodiments, the therapeutically effective amount of the anthocyanin-rich extract is about 10 g/kg body weight.

According to another embodiment, symptoms of metabolic syndrome that may be treated according to the invention include, but are not limited to, fasting hypoglycemia, high blood pressure, decreased high-density lipoprotein cholesterol, elevated triglycerides, elevated uric acid levels, and insulin resistance.

The invention also provides any of the disclosed compositions for use as a medicament.

The invention further provides a product containing the composition of any of the disclosed compositions and a second pharmaceutical agent, as a combined preparation for simultaneous, separate or sequential use as a medicament.

The invention still further provides a use of any disclosed composition for the manufacture of a medicament for the treatment of a disease in a subject.

The invention also provides a use of any disclosed composition and of a second agent for the manufacture of a medicament for the treatment of a disease in a subject.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein may also be used in the practice or testing of the present invention, the preferred methods and materials are now described. All publications mentioned herein are incorporated herein by reference to disclose and described the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention may be embodied in other specific forms without departing from the spirit or essential attributes thereof and, accordingly, reference should be made to the appended claims, rather than to the foregoing specification, as indicating the scope of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperatures, etc.) but some experimental errors and deviations should be accounted for. Unless indicated otherwise, parts are parts by weight, molecular weight is weight average molecular weight, temperature is in degrees Centigrade, and pressure is at or near atmospheric.

High-performance liquid chromatography (HPLC) is a form of column chromatography used frequently in biochemistry and analytical chemistry to separate, identify, and quantify compounds. HPLC-PDA is a form of HPLC used with herbal preparations. This technique can generate chemical spectra profile that characterizes the multicomponents of active constituents as unique characteristics. A chromatogram covering a broad range of constituents may be stored electronically and its basic pattern/profile (as reference standard) can be used for comparison with a given test sample to determine a match factor. HPLC-PDA defines the identity of active plant constituents with greater accuracy than the conventional practice of measuring selected main constituents.

### Example 1

### Anthocyanin Extraction from Berries

### 1.1. Preparation of Berry Extracts

Frozen blueberry fruits (1 kg) were blended in a mixer with 3 liters of methanol, acidified with 0.5% trifluoroacetic acid (TFA), and filtered first through muslin sheets, and subsequently through filter paper # 4, using suction. The collected hydro-alcoholic extract was evaporated to about 500 mL using a rotary evaporator at a temperature not exceeding 40° C. The concentrated aqueous extract obtained was partitioned against ethyl acetate (EtOAc) (500 mL×4) to remove lipophilic material. The aqueous layer (500 mL) was divided into two equal portions after evaporation of any remaining EtOAc. Each portion was applied on an Amberlite XAD-7 column (18×6.5 cm), preconditioned with acidified water (0.5% TFA). The columns were washed with acidified water (0.5% TFA) until the color of eluting water faded (approximately 3 liters of acidified water). The crude anthocyanin mixture was eluted with 1 liter of acidified methanol (0.5% TFA), and the combined methanol extracts were evaporated, and freeze-dried to yield 5.0 g crude anthocyanin mixture.

### 1.2. Preparation of Anthocyanin-Rich Fraction

The blueberry crude extract (5.0 g) was applied to a Sephadex LH-20 column (40×4 cm), packed in acidified water-MeOH 80:20 (0.5% TFA), and anthocyanins were eluted using the same solvent ratio. Four fractions, each approximately 200 ml, were collected when the colored material started to elute from the column. Fractions were dried down immediately using rotary evaporator and freeze-dried.

### 1.3. Quantitative Analysis of Blueberry Anthocyanins

Quantification of anthocyanins in both the crude extract and in individual fractions was performed by two different methods: by a pH differential method and by HPLC-PDA analysis methods, and were calculated as cyanidin-3-glucoside % w/w. Both methods gave nearly the same results.

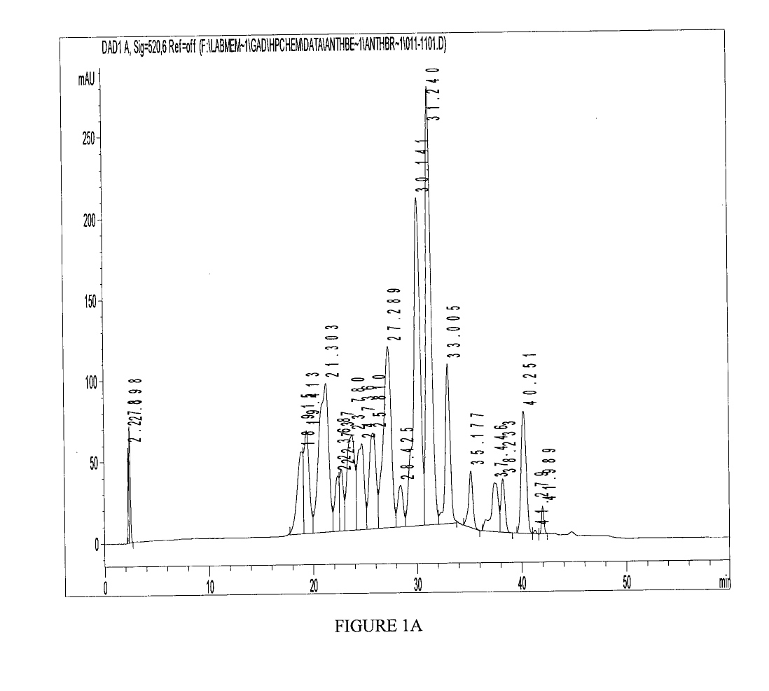

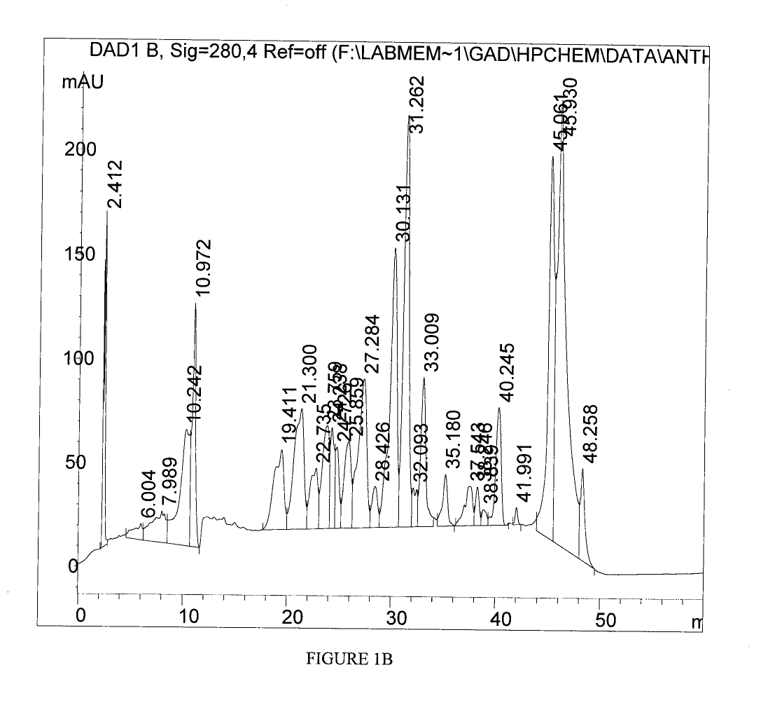

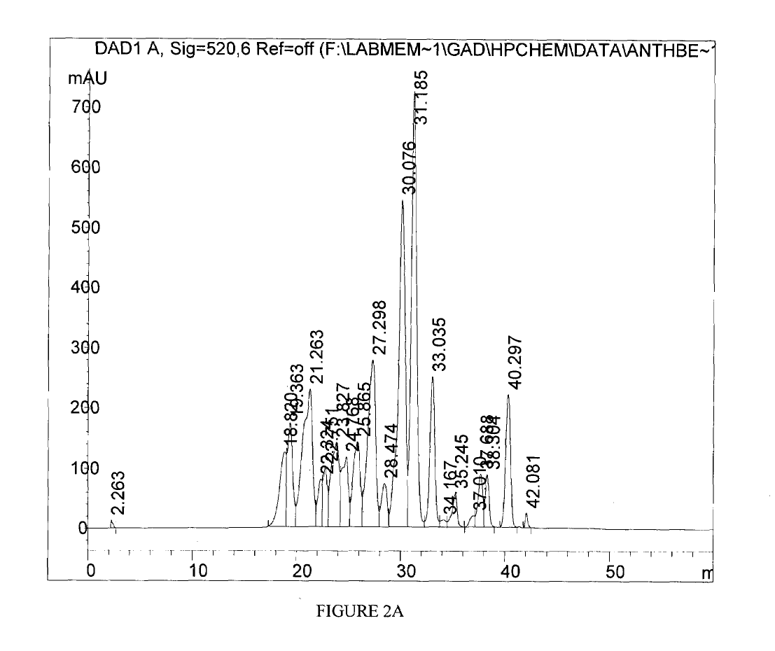

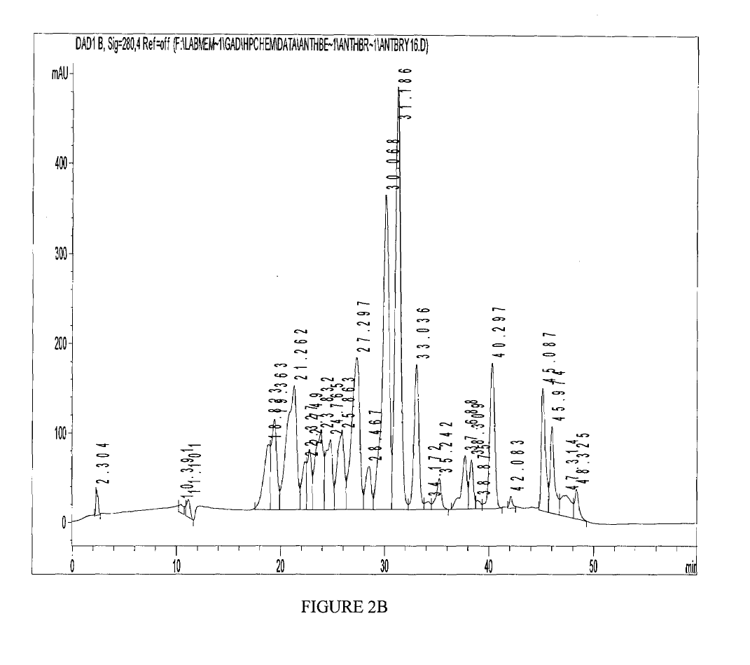

According to pH-differential method and HPLC-PDA analysis, the first fraction (Fraction 1, 1.5 g out of 5.0 g crude extract) contained the highest percentage of anthocyanins (approximately 60% w/w). The rest of the fractions (Fractions 2-4) showed decreasing percentage of anthocyanins. The crude extract showed anthocyanin percentage of approximately 30%, calculated as cyanidin-3-glucoside. HPLC profiles of the crude extract and the anthocyanin-rich fraction, Fraction 1 (designated F2), as seen in FIGS. 1A, 1B, 2A and 2B, showed the same number of individual components with increased intensity when measured at 520 nm. The HPLC-PDA profile of crude extract measured at 280 nm showed additional peaks resulted from non-anthocyanin material, while the anthocyanin-rich fractions showed decreased absorption of those additional peaks indicating the increase in anthocyanin content.

### Animal Model

The C57B6 mouse strain becomes diabetic when maintained on a high fat diet, it therefore serves as a model for Type 2 diabetes. A screen for treatments effective against diet induced diabetes utilizes male C57BL/6J mice (4-5 week old, 19-20 g) fed a high-fat diet (60% fat in kcal) for 10-16 weeks. This diet induces obesity and diabetes in this mouse strain. Extracts that induce a 20-40% decline in the blood glucose level are used for further testing.

### Example 2

### Evaluation of Antidiabetic Activity in Mice

### 2.1. Methods for Evaluating Antidiabetic Activity in Mice

Five-week-old male C57bl/6J mice were purchased from Jackson Labs (Maine) and were acclimatized for 1 week before being randomly assigned into the experimental groups. The animals were housed four to a cage with free access to water in a room with a 12:12 hour light-dark cycle (7:00 AM-7:00 PM), a temperature of 24±1° C., and the animals were weighed weekly. During the acclimatization period, each animal was fed a regular diet ad libitum. At 6-weeks old, the mice were randomly divided into two groups: low fat or very high fat fed groups. The mice continued to receive either a low fat diet (LFD) or very high-fat diet (VHFD) for a 12-week period. The high-fat diet food was purchased from Research Diets Inc., and the nutrition content of very high-fat diet food was similar to that of the low fat diet food except that carbohydrates were lower (70 kcal % vs. 20 kcal %) and the addition of more lard (10 kcal % vs. 60 kcal %) in the diet. Body weights were measured weekly, and at every other week blood was collected for blood glucose analysis. Animals fed with VHFD were fasted for 4 hours and fed orally (gavage) with plant extracts (500 mg/kg) or vehicle (metformin) to test efficacy of plant extracts. Animals fed with LFD were also fasted for 4 hours. Blood glucose readings are made 0 hours, 3 hours, and 6 hours after treatment (animals were fasted during the testing period). As a positive control, metformin (Fortamet®, Gluocphase®, Glumetza®, Romet®) was administered instead of plant extract at a dose of 300 mg/kg (MET300). Metformin is an antidiabetic drug that originates from the French lilac and has been shown to reduce symptoms of diabetes mellitus in patients. Without being limited by theory, metformin may act by increasing insulin sensitivity via increasing the utilization of glucose. The experimental protocols were approved by Rutgers University Institutional Care and Use Committee and followed federal, and state laws.

### 2.2. Blood Sampling and Plasma Assay

An oral glucose tolerance test (OGTT) was performed at the end of the treatment. On the test day, animals were fasted for 7 hours, and glucose (1.5 g/kg) then was orally administered to them. Blood glucose levels were determined from the tail vein at 0 minutes (before glucose challenge), 30 minutes, 60 minutes, and 120 minutes after glucose administration.

### 2.3. Effect of Blueberry Preparations on Blood Glucose Levels in Food Restricted C57BL6 Mice

All treatments were provided by gavage with a vehicle of 66% LABRASOL®, a lipid-based self-emulsifying excipient. Abbreviations for Table 1 are as follows: FC=crude extract, F2=anthocyanin enriched extract, FT=treated extract to promote chalcone formation, metformin is a drug used as a positive control. The results were as follows:

TABLE 1

| | | | | % decrease |
|---|---|---|---|---|
| | | | | from |
| Time (hours) | 0 hours | 2 hours | 4 hours | 0 hours |
| Vehicle | 223 ± 28 | 215 ± 16 | 223 ± 13 | 0 |
| FC (500 mg/kg) | 200 ± 9 | 191 ± 15 | 134 ± 21*^{cc} | −33 |
| F2 (500 mg/kg) | 229 ± 26 | 136 ± 29 | 111 ± 12**^{ccc} | −51 |
| FT (500 mg/kg) | 212 ± 24 | 181 ± 6 | 138 ± 10*^{c} | −35 |
| Metformin (300 | 226 ± 18 | 183 ± 25 | 165 ± 29 | −27 |
| mg/kg) | | | | |

Note that
*p < 0.05;
**p < 0.01;
***p < 0.001 vs. 0 hours;
^{c}p < 0.05;
^{cc}p < 0.01;
^{ccc}p < 0.001 vs LABRASOL ®.

Treatment with blueberry extracts formulated with the appropriate carrier effectively lowered blood glucose levels of the diabetic animals and to a greater extent than metformin (Table 1). Metformin, an approved drug used for the treatment of diabetes, was used in this study as a positive control for reference. A lower dose (300 mg/kg) of the metformin was used to compensate for differences in purity between commercially available metformin and the blueberry extracts. The blueberry extracts are mixtures of compounds and are likely to contain not only multiple compounds with hypoglycemic activity but also, and, to a greater extent, many compounds with no hypoglycemic activity. The F2 version of the extract which corresponds to the most hypoglycemic activity contained the highest concentration of anthocyanins.

### 2.4. The Effect of Blueberry Preparations on Blood Glucose Levels in Food Restricted C57BL6 Mice.

All treatments were provided by gavage with a vehicle of 66% LABRASOL®. Abbreviations for Table 2 are as follows: FC=crude extract, F2=anthocyanin-enriched extract, FT=treated extract to promote chalcone formation, metformin is a drug used as a positive control; *:p<0.05; **:p<0.01; ***:p<0.001 vs 0 hours; c:p<0.05; cc:p<0.01; ccc:p<0.001 vs LABRASOL®. The results were as follows:

TABLE 2

| | | | | % decrease |
|---|---|---|---|---|
| | | | | from |
| Time (hours) | 0 hours | 3 hours | 5 hours | 0 hours |
| Vehicle | 219 ± 20 | 213 ± 21 | 213 ± 20 | −3 |
| FC (500 mg/kg) | 236 ± 21 | 217 ± 9 | 158 ± 9*^{c} | −33 |
| F2 (500 mg/kg) | 178 ± 11 | 182 ± 8 | 125 ± 5*^{cc} | −30 |
| FT (500 mg/kg) | 251 ± 23 | 216 ± 10 | 176 ± 7* | −30 |
| F2-water (500 | 211 ± 8 | 187 ± 8 | 173 ± 22 | −18 |
| mg/kg) | | | | |
| Metformin (300 | 207 ± 18 | 211 ± 15 | 128 ± 17**^{cc} | −38 |
| mg/kg) | | | | |

The blueberry extracts were retested in the diabetic animal model yielding similar results as Example 2.3. The F2 fraction of the extract is very water soluble. The F2 fraction, in which only waster was the vehicle, was compared with the F2 fraction formulated with LABRASOL® and also was provided to animals. The F2 formulated with water was not active (p<0.05) compared to the F2 formulated with LABRASOL® and water. The LABRASOL® carrier enhances the activity of the F2 extract.

### 2.5: The Relative Effects of Maqui Berry Preparations on Blood Glucose Levels in Food Restricted C57B6 Mice.

All treatments were provided by gavage with a vehicle of 66% LABRASOL®. Abbreviations for Table 3 are as follows: FT=treated extract to promote chalcone formation, F2=anthocyanin enriched extract, MBCE=maqui berry crude extract, MBA=maqui berry anthocyanin-rich extract, metformin is a drug used as a positive control; *:p<0.05; **:p<0.01; ***:p<0.001 vs. 0 hours; c:p<0.05; cc:p<0.01; ccc:p<0.001 vs. LABRASOL®.

The results were as follows:

TABLE 3

| | | | | % decrease |
|---|---|---|---|---|
| | | | | from |
| Time (hours) | 0 hours | 3 hours | 6 hours | 0 hours |
| Vehicle | 246 ± 24 | 224 ± 10 | 214 ± 15 | −13 |
| FT (250 mg/kg) | 254 ± 10 | 239 ± 37 | 173 ± 12**^{c} | −32 |
| MBCE (500 mg/kg) | 199 ± 5 | 234 ± 42 | 173 ± 10^{c} | −13 |
| MBA (250 mg/kg) | 260 ± 19 | 194 ± 20* | 182 ± 15** | −30 |
| Metformin (250 | 249 ± 29 | 273 ± 32 | 214 ± 39 | −14 |
| mg/kg) | | | | |

FT blueberry extracts, maqui berry crude extract (MBCE) and an anthocyanin-rich preparation of the maqui berry extract (MBA) were tested for relative hypoglycemic activity (Table 3). The berry FT extracts were retested as described above, but at the lower dose of 250 mg/kg. The FT extract (250 mg/kg) lowered blood glucose more effectively than metformin (250 mg/kg). The MBA extract was more than twice as active as the metformin control when provided at the same concentration as the blueberry FT extract.

### Example 3

### Hypoglycemic Effect of Delphinidin-3-Glucoside and Malvidin-3-Glucoside Relative to Blueberry Anthocyanin-Enriched Extracts and Metformin in Insulin Resistant C57bl/6J Mice

The hypoglycemic effects of delphinidin-3-glucoside (Dp 3-glc) and malvidin-3-glucoside (Mv-3-glc) relative to blueberry anthocyanin-enriched extracts and metformin in insulin resistant C57bl/6J mice were investigated. Malvidin glycosides are the predominant anthocyanins of the lowbush blueberry extracts. Delphinidin-3-glucoside likely is a major component of the blueberry extracts. It is reported to be the active hypoglycemic compound from the leaves of Vaccinium species. The animals were maintained on a high fat diet and food restricted 4 hours prior to treatment by gavage. The extracts and metformin were formulated with 66% Labrasol as the delivery vehicle

Our results showed that malvidin-3-glucoside had hypoglycemic activity at a dose of 300 mg/kg that was comparable to the hypoglycemic activity of the phenolics-rich extract at a dose of 500 mg/kg (Table 4). The delphinidin-3-glucoside, however, did not significantly show hypoglycemic activity.

TABLE 4

| | | | % decrease |
|---|---|---|---|
| Treatment | 0 hours | 6 hours | from 0 h |
| Vehicle | 229 ± 16.3 | 213.2 ± 19 | −6 ± 3.6 |
| Dp 3-glc (300 mg/kg) | 214 ± 7.4 | 186.6 ± 15.5 | −17 ± 5.7 |
| Mv-3-glc (300 mg/kg) | 204 ± 20.8 | 134.4 ± 8.3 | −31 ± 6.3 |
| Anthocyanin enriched | 210.3 ± 9.5 | 131.3 ± 7.9 | −36 ± 3.4 |
| fraction (500 mg/kg)d | | | |
| Metformin (300 mg/kg) | 206.7 ± 11 | 108 ± 8.9 | −49 ± 4.5 |

Note
*p < 0.05;
**p < 0.01;
***p < 0.001 vs. 0 hour.

Malvidin-3-O-glucoside treatment (300 mg/kg) by gavage with Labrasol® lowered blood glucose levels in the mice by 34%, while the same treatment with delphinidin-3-O-glucoside did not have significant hypoglycemic activity. The hypoglycemic activity of malvidin glucoside was comparable to the metformin positive control at 300 mg/kg and the anthocyanin extracts extract at 500 mg/kg. The malvidin glucoside concentration in the anthocyanin-enriched extract was only about 10%, thus providing an effective dose of 50 mg/kg for the malvidin glucoside. The hypoglycemic activity observed for the anthocyanin-enriched extract may result from the combined activity of additional components acting together with the malvidin-3-O-glucoside. The delphinidin-3-O-glucoside, which is not active when delivered as a pure compound, appears to contribute significantly less to the hypoglycemic activity of the anthocyanin-enriched extract. This observation is not consistent with early reports naming myrtillin, (another name for delphinidin-3-O-glucoside) as the hypoglycemic compound from the leaves of Vaccinium species (Murray, 1997).

### Example 4

### Relative Effects of Maqui Berry Anthocyanin-Rich Extract on Blood Glucose Levels in Food Restricted C57B6 Mice Using Water as the Gavage Vehicle

Maqui berry extract (MBA) was tested using water as the vehicle as shown in Table 5. The MBA was active at both 500 mg/kg and 250 mg/kg. Therefore, the use of Labrasol as a bioenhancing agent dose was not necessary with the MBA. A robust response was seen when Labrasol was not included in the dosing vehicle.

TABLE 5

| | | | % decrease |
|---|---|---|---|
| Treatment | 0 hours | 6 hours | from 0 hours |
| Water | 198.4 ± 6.8 | 188.0 ± 7.6 | −4.4 ± 5.1 |
| MBA (500 mg/kg) | 194.7 ± 9 | 124.2 ± 6.2 | −35.3 ± 5.1 |
| MBA (250 mg/kg) | 194.5 ± 7.5 | 147.2 ± 5 | −23.6 ± 4.5 |
| Metformin-(300 mg/kg | 189 ± 11.1 | 106.8 ± 14.2 | −43.4 ± 7.8 |

### SUMMARY

Extracts were prepared from frozen blueberry fruit using different methods to create related extracts with different levels of anthocyanin enrichment. The extracts were formulated with LABRASOL®, a pharmaceutical excipient, and used to treat mice made diabetic by being maintained on a high fat diet. Prior to treatment, the mice were food restricted for 4 hours Treatment was provided by gavage, and blood glucose measurements were taken.

All of the blueberry extracts lowered blood glucose levels in the diabetic mice. The experiments were repeated three additional times, each yielding similar results. The anthocyanin rich fraction F2 was the most active preparation. It lowered blood glucose levels greater than the drug metformin, which was included as a positive control. The activity of this fraction was enhanced when formulated with LABRASOL®.

The results indicate that anthocyanin-rich extracts of blueberries and maqui berries are hypoglycemic agents. Blueberries and maqui berries contain different individual anthocyanins. The known anthocyanins of blueberry are: delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; malvidin-6-acetyl-3-glucoside.

The known anthocyanins of maqui berries are: delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; cyanidin 3-glucoside.

We claim:

1. A method for treating at least one symptom of a disordered metabolism syndrome in a subject, the method comprising the steps:
(a) isolating a disordered metabolism syndrome modifying agent derived from a berry comprising an anthocyanin-rich extract;
(b) administering a therapeutically effective amount of a composition comprising
(i) the isolated hyperglycemia-modifying agent to a subject in need thereof and
(ii) a pharmaceutically acceptable carrier,
thereby treating the symptoms of the disordered metabolism syndrome.

2. The method according to claim 1, wherein the disordered metabolism syndrome is diabetes.

3. The method according to claim 1, wherein the disordered metabolism syndrome is metabolic syndrome.

4. The method according to claim 1, wherein the berry is a blueberry.

5. The method according to claim 4, wherein the blueberry is a wild lowbush blueberry.

6. The method according to claim 4, wherein the blueberry is a fruit of the plant genus Vaccinium.

7. The method according to claim 1, wherein the berry is a maqui berry fruit of the plant genus Aristotelia.

8. The method according to claim 1, wherein the berry is an elderberry or a chokeberry or a blackcurrant.

9. The method according to claim 1, wherein the therapeutically effective amount is from about 0.001 mg/kg body weight to about 10 g/kg body weight.

10. The method according to claim 1, wherein the anthocyanin-rich extract comprises at least one of delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; and malvidin-6-acetyl-3-glucoside.

11. The method according to claim 1, wherein the anthocyanin-rich extract comprises at least one of delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; and cyanidin 3-glucoside.

12. The method according to claim 1, wherein the anthocyanin-rich extract is an aqueous extract.

13. The method according to claim 1, wherein the anthocynanin-rich extract comprises at least one compound of the structure: 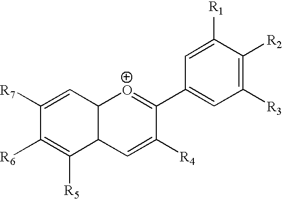
wherein
R₁, R₂, R₃, R₄, R₅, R₆ and R₇ each independently is —H, —OH, —OCH₃, a monosaccharide, a disaccharide or a polysaccharide.

14. The method according to claim 13, wherein R₁ and R₃ are —H; and R₂, R₄, R₅, R₆ and R₇ are —OH.

15. The method according to claim 13, wherein R₃ and R₆ are —H and R₁, R₂, R₄, R₅ and R₇ are —OH.

16. The method according to claim 13, wherein R₆ is —H, and R₁, R₂, R₃, R₄, R₅ and R₇ are —OH.

17. The method according to claim 13, wherein R₁ and R₅ are —OCH₃, and R₂, R₃, R₄ and R₇ are —OH, and R₆ is —H.

18. The method according to claim 13, wherein R₁, R₂, R₅ and R₇ are —OH, and R₃, R₄ and R₆ are —H.

19. The method according to claim 13, wherein R₁, R₃ and R₆ are —H, and R₂, R₄, R₅ and R₇ are —OH.

20. The method according to claim 13, wherein R₁ and R₃ are —OCH₃, and R₂, R₄, R₅ and R₇ are —OH, and R₆ is —H.

21. The method according to claim 13, wherein R₁ is —OCH₃, R₂, R₄, R₅ and R₇ are —OH, R₃ and R₆ are —H.

22. The method according to claim 13, wherein R₁, R₂, R₄, R₅ and R₇ are —OH; R₃ is —OCH₃, and R₆ is —H.

23. The method according to claim 13, wherein R₁ and R₇ are —OCH₃, R₂, R₄ and R₅ are —OH, and R₃ and R₆ are —H.

24. The method according to claim 1, wherein the pharmaceutically acceptable carrier is an esterified glyceride.

25. The method according to claim 24, wherein the esterified glyceride comprises at least one of polyethylene glycol or fatty acids.

26. The method according to claim 1, wherein the method decreases hyperglycemia by from about 5% to about 75% relative to a healthy subject.

27. The method according to claim 1, wherein the composition comprises a nutritional supplement.

28. The method according to claim 1, wherein the composition comprises a comestible.

29. The method according to claim 28, wherein the comestible is a beverage.

30. The method according to claim 1, wherein the hyperglycemia-modifying agent is isolated from the berry by extracting a berry homogenate with a polar solvent.

31. The method according to claim 1, wherein the hyperglycemia-modifying agent is soluble in a polar solvent.

32. The method according to claim 1, wherein the composition is administered orally, buccally, parenterally, topically, by inhalation or insufflation, or rectally.

33. The method according to claim 1, wherein the composition is a pharmaceutical composition.

34. A composition for the treatment of a disordered metabolism syndrome comprising (a) a therapeutically effective amount of a disordered metabolism syndrome modifying agent derived from a berry comprising an anthocyanin-rich extract; and (b) a pharmaceutically acceptable carrier.

35. The composition according to claim 34, wherein the berry is a blueberry.

36. The composition according claim 35, wherein the blueberry is a wild lowbush blueberry.

37. The composition according to claim 35, wherein the blueberry is a fruit of the plant genus Vaccinium.

38. The composition according to claim 34, wherein the berry is a maqui berry fruit of the plant genus Aristotelia.

39. The pharmaceutical composition according to claim 34, wherein the berry is an elderberry or a chokeberry or a blackcurrant.

40. The composition according to claim 34, wherein the therapeutically effective amount is from about 0.001 mg/kg body weight to about 10 g/kg body weight.

41. The composition according to claim 34, wherein the anthocyanin-rich extract comprises at least one of delphinidin-3-galactoside; delphindin-3-glucoside; cyanidin-3-galactoside; delphinidin-3-arabinoside; cyanidin-3-glucoside; petunidin-3-galactoside; petunidin-3-glucoside; cyanidin-3-arabinoside; peonidin-3-galactoside; perunidin-3-arabinoside; malvidin-3-galactoside; peonidin-3-glucoside; malvidin-3-glucoside; peonidin-3-arabinoside; malvidin-3-arabinoside; delphinidin-6-acetyl-3-glucoside; cyanidin-6-acetyl-3-glucoside; malvidin-6-acetyl-3-galactoside; petunidin-6-acetyl-3-glucoside; peonidin-6-acetyl-3-glucoside; and malvidin-6-acetyl-3-glucoside.

42. The composition according to claim 34, wherein the anthocyanin-rich extract comprises at least one of delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; and cyanidin 3-glucoside.

43. The composition according to claim 34, wherein the anthocyanin-rich extract is an aqueous extract.

44. The composition according to claim 34, wherein the anthocynanin-rich extract comprises at least one compound of the structure: 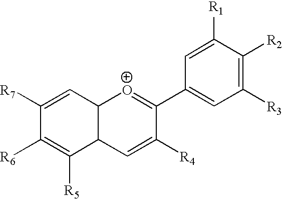
wherein
R₁, R₂, R₃, R₄, R₅, R₆ and R₇ each independently is —H, —OH, —OCH₃, a monosaccharide, a disaccharide or a polysaccharide.

45. The composition according to claim 44, wherein R₁ and R₃ are —H; and R₂, R₄, R₅, R₆ and R₇ are —OH.

46. The composition according to claim 44, wherein R₃ and R₆ are —H and R₁, R₂, R₄, R₅ and R₇ are —OH.

47. The composition according to claim 44, wherein R₆ is —H, and R₁, R₂, R₃, R₄, R₅ and R₇ are —OH.

48. The composition according to claim 44, wherein R₁ and R₅ are —OCH₃, and R₂, R₃, R₄ and R₇ are —OH, and R₆ is —H.

49. The composition according to claim 44, wherein R₁, R₂, R₅ and R₇ are —OH, and R₃, R₄ and R₆ are —H.

50. The composition according to claim 44, wherein R₁, R₃ and R₆ are —H, and R₂, R₄, R₅ and R₇ are —OH.

51. The composition according to claim 44, wherein R₁ and R₃ are —OCH₃, and R₂, R₄, R₅ and R₇ are —OH, and R₆ is —H.

52. The composition according to claim 44, wherein R₁ is —OCH₃, R₂, R₄, R₅ and R₇ are —OH, R₃ and R₆ are —H.

53. The composition according to claim 44, wherein R₁, R₂, R₄, R₅ and R₇ are —OH; R₃ is —OCH₃, and R₆ is —H.

54. The composition according to claim 44, wherein R₁ and R₇ are —OCH₃, R₂, R₄ and R₅ are —OH, and R₃ and R₆ are —H.

55. The composition according to claim 44, wherein the pharmaceutically acceptable carrier is an esterified glyceride.

56. The composition according to claim 55, wherein the esterified glyceride comprises at least one of polyethylene glycol or fatty acids.

57. The composition according to claim 34, wherein the composition is a pharmaceutical composition.

58. The pharmaceutical composition according to claim 34, wherein the method decreases hyperglycemia from about 5% to about 75% relative to a healthy subject.

59. The method according to claim 34, wherein the composition comprises a nutritional supplement.

60. The method according to claim 34, wherein the composition comprises a comestible.

61. The method according to claim 60, wherein the comestible is a beverage.

62. The method according to claim 34, wherein the disordered metabolism syndrome modifying agent is isolated from the berry by extracting a berry homogenate with a polar solvent.

63. The method according to claim 34, wherein the disordered metabolism syndrome modifying agent is soluble in a polar solvent.

64. The method according to claim 34, wherein the composition is administered orally, buccally, parenterally, topically, by inhalation or insufflation, or rectally.
